(19) United States
(12) Patent Application Publication
(10) Pub. No.: US 2026/0076970 A1
(43) Pub. Date: Mar. 19, 2026

(54) CANCER TREATMENT USING MTDP INHIBITORS AND PLK1 INHIBITORS

(71) Applicant: Cardiff Oncology, Inc., San Diego, CA (US)

(72) Inventors: Maya Ridinger, San Diego, CA (US); Tod Smeal, San Diego, CA (US); Mark Erlander, San Diego, CA (US)

(21) Appl. No.: 19/109,426

(22) PCT Filed: Sep. 8, 2023
(86) PCT No.: PCT/US2023/073692

§ 371 Date: Mar. 6, 2025

Related U.S. Application Data
(60) Provisional application No. 63/405,308, filed on Sep. 9, 2022.

(51) Int. Cl. A61K 31/519; A61K 31/337; A61P 35/00

(52) U.S. Cl. CPC A61K 31/519; A61K 31/337; A61P 35/00

(57) ABSTRACT

Provided include methods, compositions and kits for treating cancer in a subject. The method can comprise administrating an MTDP inhibitor (for example, paclitaxel) and a PLK1 inhibitor (for example, onvansertib) to the subject in a manner sufficient to inhibit or reduce progression of the cancer.

### RELATED APPLICATIONS

The present application is a U.S. national phase application under 35 U.S.C. § 371 of International Application No. PCT/US2023/073692, filed on Sep. 8, 2023 and published as WO 2024/054950 A1 on Mar. 14, 2024, which claims priority under 35 U.S.C. § 119(e) to U.S. Provisional Application No. 63/405,308, filed Sep. 9, 2022. The content of each of these related applications is incorporated herein by reference in its entirety.

### BACKGROUND

### Field

The present application generally relates to treatment for cancer. More specifically, combination therapies for treating cancer using microtubule depolymerization (MTDP) inhibitors in combination with polo-like kinase 1 (PLK1) inhibitors are provided.

### Description of the Related Art

PLK1 is a serine/threonine kinase and the most well characterized member of this family of 5 closely related regulatory proteins. PLK-1 is a master regulator of mitosis via its control of the entry and progression of cells into and through mitosis. PLK1 performs several important functions throughout mitotic (M) phase of the cell cycle, including the regulation of centrosome maturation and spindle assembly, the removal of cohesins from chromosome arms, the inactivation of anaphase-promoting complex/cyclosome (APC/C) inhibitors, and the regulation of mitotic exit and cytokinesis. PLK1 plays a key role in centrosome functions and the assembly of bipolar spindles. PLK1 controls kinetochore interactions with the spindle microtubules that is required for successful separation and segregation chromatids to the appropriate mother and daughter cells. PLK1 also acts as a negative regulator of p53 family members leading to ubiquitination and subsequent degradation of p53/TP53, inhibition of the p73/TP73 mediated pro-apoptotic functions and phosphorylation/degradation of bora, a cofactor of Aurora kinase A. During the various stages of mitosis PLK1 localizes to the centrosomes, kinetochores and central spindle. PLK1 is aberrantly overexpressed in a variety of human cancers and is correlated with cellular proliferation and poor prognosis.

Tubulin targeting agents is a diverse class of compounds that can bind to the tubulin dimer. Compounds include natural or synthetic compounds that can bind to the taxane site, colchicine domain, Vinca domain, or another domain of the tubulin dimer. The compounds can affect microtubule dynamics and/or polymerization. Some are strong antimitosis agents that lead to apoptosis and are candidates for cancer therapeutics. Examples of tubulin targeting agents include MTDP inhibitors, such as paclitaxel, docetaxel, epothilones, ixabepilone, and discodermolide.

There is a need to develop effective treatment for cancer patients, including the patients resistant to MTDP inhibitor treatment.

### SUMMARY

Provided include methods, compositions and kits for treating cancer. Some embodiments provide a method of treating cancer, where the method comprises: administrating a microtubule depolymerization (MTDP) inhibitor and a Polo-like kinase 1 (PLK1) inhibitor to a subject with cancer, thereby inhibiting or reducing progression of the cancer in the subject. In some embodiments, the subject has hematologic or solid cancers. The cancer can be breast cancer, where the breast cancer can be invasive breast cancer, unresectable breast carcinoma, locally advanced breast cancer, metastatic breast cancer, inflammatory breast cancer, triple negative breast cancer (TNBC), hormone receptor/growth factor receptor-negative breast cancer, HER2-negative breast cancer, and/or hormone receptor negative breast carcinoma. In some embodiments, the subject has TNBC. In some embodiments, the method comprises identifying a subject with cancer as a subject having more than about 1000 neutrophils per mm³, more than about 100,000 per mm³ platelets, about 1.5 times institutional upper limit normal of total bilirubin, and either less than about 1.5 times institutional upper limit normal or 60 mL/min glomerular filtration rate of serum creatinine, or a combination thereof. In some embodiments, the subject has more than about 1000 neutrophils per mm³, more than about 100,000 per mm³ platelets, about 1.5 times institutional upper limit normal of total bilirubin, and either less than about 1.5 times institutional upper limit normal or 60 mL/min glomerular filtration rate of serum creatinine.

The PLK1 inhibitor and the MTDP inhibitor can be co-administered simultaneously. The PLK1 inhibitor and the MTDP inhibitor can also be administered sequentially. In some embodiments, the administration of the PLK1 inhibitor is orally, wherein prior to the administration, the subject fasts for more than about 30 minutes, and wherein after the administration, the subject fasts for about 4 hours. In some embodiments, the administration of the MTDP inhibitor is intravenously, optionally through an in-line filter with a microporous membrane not greater than 0.22 micron, wherein prior to administration, the subject is administered (i) about 20 mg of dexamethasone orally at about 12 hours and about 6 hours prior or about 12 mg of dexamethasone intravenously at about 60 minutes prior; (ii) about 50 mg of diphenhydramine intravenously at from about 30 minutes to 60 about minutes prior; and/or (iii) about 300 mg of cimetidine intravenously at from about 30 minutes to about 60 minutes prior or about 20 mg of famotidine intravenously from about 30 minutes to about 60 minutes prior.

The inhibition of cancer progression can be greater than the combined inhibition of progression caused by the MTDP inhibitor alone plus the PLK1 inhibitor alone. In some embodiments, the subject achieves a complete response. In some embodiments, the subject has received a prior MTDP inhibitor or PLK1 inhibitor treatment. In some embodiments, the subject did not respond to treatment with the MTDP inhibitor or PLK1 inhibitor alone. In some embodiments, the subject is known to be resistant to an MTDP inhibitor or PLK1 inhibitor therapy. In some embodiments, the MTDP inhibitor and the PLK1 inhibitor are each administered to the subject in a cycle of 28 days. In some embodiments, the MTDP inhibitor is administered to the subject in a cycle of 28 days and PLK1 inhibitor is administered to the subject in a cycle of 28 days, comprising from about 14-28 days of administration and from about 0-14 days of non-administration, preferably from about 15-27 days of administration and from about 1-13 days of non-administration, preferably from about 16-26 days of administration and from about 2-12 days of non-administration, preferably from about 17-25 days of administration and from about 3-11 days of non-administration, preferably from about 18-24 days of administration and from about 4-10 days of non-administration, preferably from about 19-23 days of administration and from about 5-9 days of non-administration, preferably from about 20-22 days of administration and from about 6-8 days of non-administration, preferably from about 21 days of administration and from about 7 days of non-administration, and preferably the administration days are consecutive. In some embodiments, the MTDP inhibitor, the PLK1 inhibitor, or both are administered in a cycle of 28 days.

Each cycle of treatment can be, or be at least about, 28 days. In some embodiments, each cycle of treatment is from about 14 days to about 28 days. The PLK1 inhibitor can be administered on at least four days in the cycle. In some embodiments, the PLK1 inhibitor is not administered on at least one day in the cycle. In some embodiments, the MTDP inhibitor is administered once daily or twice daily. In some embodiments, the MTDP inhibitor is administered once daily. In some embodiments, subject undergoes at least two cycles of the administration of the MTDP inhibitor and the PLK1 inhibitor. MTDP inhibitor can be, for example, paclitaxel, docetaxel, acetyltaxol, paclitaxel; lutetium Lu 177 vipivotide tetraxetan; 7-hexanoyltaxol; cabazitaxel; larotaxel; milataxel; ortataxel; tesetaxel; taxoprexin; opaxio; taxoprexin (DHA-paclitaxel); Poly(L-glutamic acid)-paclitaxel; abraxane; SB-T-1214; SB-T1216; SB-T121602; SB-T-12854; DHA-SB-T1214; abeo-taxanes, wherein the abeo-taxanes is optionally abeo-taxane 15a.2; docetaxl-d9-t-Boc; docetaxel-f3-t-Boc; cabazitaxel-7,10-d6; Poly(glutamyl-glutamate)-taxane conjugates; or a pharmaceutically acceptable salt, hydrate, solvate or prodrug thereof; or any combinations thereof. In some embodiments, MTDP inhibitor is paclitaxel. In some embodiments, from about 48 mg/m² of body surface area to about 80 mg/m² of body surface area of paclitaxel is administered to the subject.

PLK1 inhibitor can be, for example, onvansertib (NMS-P937), BI2536, volasertib (BI 6727), GSK461364, adavosertib (AZD1775), CYC140, HIMIN-176, HMN-214, rigosertib (ON-01910), MLN0905, TKM-080301, TAK-960, GTPL10072, Ro3280; or a pharmaceutically acceptable salt, hydrate, solvate or prodrug thereof; and any combinations thereof. In some embodiments, the PLK1 inhibitor is onvansertib. In some embodiments, onvansertib is administered at a dose from about 6 mg/m² of body surface area to about 24 mg/m² of body surface area. In some embodiments, onvansertib is administered at a dose of 9 mg/m² of body surface area, 12 mg/m² of body surface area, and 24 mg/m² of body surface area.

In some embodiments, the subject has received at least one prior cancer treatment. In some embodiments, the prior treatment does not comprise the use of a MTDP inhibitor, a PLK1 inhibitor, or both; and optionally the PLK1 inhibitor is onvansertib. In some embodiments, the subject was in remission for cancer, for example in complete remission (CR) or in partial remission (PR). In some embodiments, the method further comprises determining cancer status of the subject. The method can, for example, further comprises determining responsiveness of the subject to the treatment of the MTDP inhibitor and the PLK1 inhibitor. In some embodiments, the method further comprises administering one or more cancer therapeutics or therapies for the cancer. In some embodiments, the subject is human.

Also disclosed herein includes a method of sensitizing cancer cells to a microtubule depolymerization (MTDP) inhibitor, the method comprising: contacting cancer cells with a composition comprising a Polo-like kinase 1 (PLK1) inhibitor, thereby sensitizing the cancer cells to the MTDP inhibitor. In some embodiments, the PLK1 inhibitor is onvansertib and/or MTDP inhibitor is paclitaxel. The method can comprise contacting cancer cells with the composition occurs in vitro, ex vivo, and/or in vivo. In some embodiments, contacting cancer cells with the composition is in a subject. In some embodiments, the subject did not respond to, or is known to be resistant to, the MTDP inhibitor. In some embodiments, the subject had prior treatment with the MTDP inhibitor. The subject can be a mammal, for example a human. The method can comprise determining sensitization of the cancer cells to the MTDP inhibitor after being contacted with the composition.

In some embodiments, the method comprises contacting the cancer cells with the MTDP inhibitor. Contacting the cancer cells with the MTDP inhibitor can occur in the subject. The method can comprise determining the response of the subject to the MTDP inhibitor. In some embodiments, contacting the cancer cells with the MTDP inhibitor is concurrent with the contacting the cancer cells with the composition, or after the contacting the cancer cells with the composition. In some embodiments, the subject having more than about 1000 neutrophils per mm³, more than about 100,000 per mm³ platelets, about 1.5 times institutional upper limit normal of total bilirubin, and either less than about 1.5 times institutional upper limit normal or 60 mL/min glomerular filtration rate of serum creatinine.

Also disclosed includes a kit, comprising: a Polo-like kinase 1 (PLK1) inhibitor; and a manual providing instructions for co-administrating the PLK1 inhibitor with a microtubule depolymerization (MTDP) inhibitor to a subject in need thereof for treating cancer. The cancer can be, for example, hematologic or solid cancers, and optionally the cancer is breast cancer, wherein the breast cancer is optionally invasive breast cancer, unresectable breast carcinoma, locally advanced breast cancer, metastatic breast cancer, inflammatory breast cancer, triple negative breast cancer (TNBC), hormone receptor/growth factor receptor-negative breast cancer, HER2-negative breast cancer, and/or hormone receptor negative breast carcinoma. In some embodiments, the cancer is TNBC. In some embodiments, the subject has more than about 1000 neutrophils per mm³, more than about 100,000 per mm³ platelets, about 1.5 times institutional upper limit normal of total bilirubin, and either less than about 1.5 times institutional upper limit normal or 60 mL/min glomerular filtration rate of serum creatinine.

In some embodiments, the PLK1 inhibitor is onvansertib and/or the MTDP inhibitor is paclitaxel. The instructions can comprise instructions for co-administrating the PLK1 inhibitor and the MTDP inhibitor simultaneously. In some embodiments, the instructions comprise instructions for co-administrating the PLK1 inhibitor and the MTDP inhibitor sequentially. In some embodiments, the instructions comprise instructions for administering of the PLK1 inhibitor orally, wherein prior to the administration, the subject fasts for more than about 30 minutes, and wherein after the administration, the subject fasts for about 4 hours.

In some embodiments, the instructions comprise instructions for administrating the MTDP inhibitor intravenously, preferably through an in-line filter with a microporous membrane not greater than 0.22 micron, wherein prior to administration, the subject is administered (i) about 20 mg of dexamethasone orally at about 12 hours and about 6 hours prior or about 12 mg of dexamethasone intravenously at about 60 minutes prior; (ii) about 50 mg of diphenhydramine intravenously at from about 30 minutes to 60 about minutes prior; and (iii) about 300 mg of cimetidine intravenously at from about 30 minutes to about 60 minutes prior or about 20 mg of famotidine intravenously from about 30 minutes to about 60 minutes prior.

In some embodiments, the instructions comprise instructions the subject has received a prior MTDP inhibitor or PLK1 inhibitor treatment. In some embodiments, the instructions comprise instructions the subject did not respond to treatment with the MTDP inhibitor or PLK1 inhibitor alone. In some embodiments, the instructions comprise instructions the subject is known to be resistant to an MTDP inhibitor or PLK1 inhibitor therapy. In some embodiments, the instructions comprise instructions for administering each of the paclitaxel and onvansertib to the subject in a cycle of 28 days. In some embodiments, the instructions comprise instructions for administering each of the paclitaxel and onvansertib to the subject in a cycle of at least five times within a week. In some embodiments, the instructions comprise instructions for administering the MTDP inhibitor, onvansertib, or both are in a cycle of at least 7 days. In some embodiments, each cycle of treatment is at least about 21 days. In some embodiments, each cycle of treatment is from about 14 days to about 28 days. In some embodiments, the instructions comprise instructions for administering onvansertib for 28 days, comprising from about 14-28 days of administration and from about 0-14 days of non-administration, preferably from about 15-27 days of administration and from about 1-13 days of non-administration, preferably from about 16-26 days of administration and from about 2-12 days of non-administration, preferably from about 17-25 days of administration and from about 3-11 days of non-administration, preferably from about 18-24 days of administration and from about 4-10 days of non-administration, preferably from about 19-23 days of administration and from about 5-9 days of non-administration, preferably from about 20-22 days of administration and from about 6-8 days of non-administration, preferably from about 21 days of administration and from about 7 days of non-administration, and preferably the administration days are consecutive. In some embodiments, the instructions comprise instructions for not administering onvansertib on at least one day in the cycle. In some embodiments, the instructions comprise instructions for administrating the MTDP inhibitor for 28 days. The MTDP inhibitor can be a reversible MTDP inhibitor. In some embodiments, the MTDP inhibitor is paclitaxel or a pharmaceutically acceptable salt, hydrate, solvate or prodrug thereof.

In some embodiments, the instructions comprise instructions for administering onvansertib at a dose of from about 3 mg/m² of body surface area to about 24 mg/m² of body surface area and/or administering paclitaxel at a dose of from about 48 mg/m² of body surface area to about 80 mg/m² of body surface area. In some embodiments, onvansertib is administered at a dose of 9 mg/m² of body surface area, 12 mg/m² of body surface area, and 24 mg/m² of body surface area. In some embodiments, the subject has received at least one prior treatment for the cancer. In some embodiments, the prior treatment does not comprise the use of a MTDP inhibitor, onvansertib, or both. In some embodiments, the subject was in remission for cancer, for example in complete remission (CR) or in partial remission (PR). In some embodiments, the kit further comprises the MTDP inhibitor, preferably paclitaxel; and/or PLK1 inhibitor, preferably onvansertib

### BRIEF DESCRIPTION OF THE DRAWINGS

FIG. 1 depicts non-limiting exemplary embodiments and data related to a schematic of the dose escalation decision map of the Phase 1b Trial with BOIN design.

FIG. 2 depicts non-limiting exemplary embodiments and data related to a schematic of the Phase 2 Trial.

FIG. 3 depicts non-limiting exemplary embodiments and data related to the Combination Index (CI) for the combination paclitaxel-onvansertib in cell lines with mutated p53 (left) and wild type p53 (right).

FIG. 4 depicts non-limiting exemplary embodiments and data related to the in vivo efficacy of onvansertib (O) alone or in combination with paclitaxel (P) against SUM159 xenografts.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, a “subject” refers to an animal that is the object of treatment, observation or experiment. “Animals” include cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes, and, in particular, humans.

As used herein, a “patient” refers to a subject that is being treated by a medical professional, such as a Medical Doctor (i.e., Doctor of Allopathic medicine or Doctor of Osteopathic medicine) or a Doctor of Veterinary Medicine, to attempt to cure, or at least ameliorate the effects of, a particular disease or disorder or to prevent the disease or disorder from occurring in the first place. In some embodiments, the patient is a human or an animal. In some embodiments, the patient is a mammal.

As used herein, “administration” or “administering” refers to a method of giving a dosage of a pharmaceutically active ingredient to a vertebrate.

As used herein, a “dosage” refers to the combined amount of the active ingredients (e.g., taxane-derived diterpenoids, including paclitaxel, and onvansertib).

As used herein, a “unit dosage” refers to an amount of therapeutic agent administered to a patient in a single dose.

As used herein, the term “daily dose” or “daily dosage” refers to a total amount of a pharmaceutical composition or a therapeutic agent that is to be taken within 24 hours.

As used herein, the term “delivery” refers to approaches, formulations, technologies, and systems for transporting a pharmaceutical composition or a therapeutic agent into the body of a patient as needed to safely achieve its desired therapeutic effect. In some embodiments, an effective amount of the composition or agent is formulated for delivery into the blood stream of a patient.

As used herein, the term “formulated” or “formulation” refers to the process in which different chemical substances, including one or more pharmaceutically active ingredients, are combined to produce a dosage form. In some embodiments, two or more pharmaceutically active ingredients can be co-formulated into a single dosage form or combined dosage unit, or formulated separately and subsequently combined into a combined dosage unit. A sustained release formulation is a formulation which is designed to slowly release a therapeutic agent in the body over an extended period of time, whereas an immediate release formulation is a formulation which is designed to quickly release a therapeutic agent in the body over a shortened period of time.

As used herein, the term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile.

As used herein, the term “pharmaceutically acceptable carrier” refers to pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to a diseased tissue or a tissue adjacent to the diseased tissue. Carriers or excipients can be used to produce compositions. The carriers or excipients can be chosen to facilitate administration of a drug or pro-drug. Examples of carriers include calcium carbonate, calcium phosphate, various sugars such as lactose, glucose, or sucrose, or types of starch, cellulose derivatives, gelatin, vegetable oils, polyethylene glycols and physiologically compatible solvents. Examples of physiologically compatible solvents include sterile solutions of water for injection (WFI), saline solution, and dextrose.

As used herein, the term “pharmaceutically acceptable salt” refers to any acid or base addition salt whose counter-ions are non-toxic to the patient in pharmaceutical doses of the salts. A host of pharmaceutically acceptable salts are well known in the pharmaceutical field. If pharmaceutically acceptable salts of the compounds of this disclosure are utilized in these compositions, those salts are preferably derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate, undecanoate, hydrohalides (e.g., hydrochlorides and hydrobromides), sulphates, phosphates, nitrates, sulphamates, malonates, salicylates, methylene-bis-b-hydroxynaphthoates, gentisates, isethionates, di-p-toluoyltartrates, ethanesulphonates, cyclohexylsulphamates, quinates, and the like. Pharmaceutically acceptable base addition salts include, without limitation, those derived from alkali or alkaline earth metal bases or conventional organic bases, such as triethylamine, pyridine, piperidine, morpholine, N-methylmorpholine, ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

As used herein, the term “hydrate” refers to a complex formed by combination of water molecules with molecules or ions of the solute. As used herein, the term “solvate” refers to a complex formed by combination of solvent molecules with molecules or ions of the solute. The solvent can be an organic compound, an inorganic compound, or a mixture of both. Solvate is meant to include hydrate, hemi-hydrate, channel hydrate and the likes. Some examples of solvents include, but are not limited to, methanol, N,N-dimethylformamide, tetrahydrofuran, dimethylsulfoxide, and water.

As used herein, “therapeutically effective amount” or “pharmaceutically effective amount” refers to an amount of therapeutic agent, which has a therapeutic effect. The dosages of a pharmaceutically active ingredient which are useful in treatment when administered alone or in combination with one or more additional therapeutic agents are therapeutically effective amounts. Thus, as used herein, a therapeutically effective amount refers to an amount of therapeutic agent which produces the desired therapeutic effect as judged by clinical trial results and/or model animal studies. The therapeutically effective amount will vary depending on the compound, the disease, disorder or condition and its severity and the age, weight, etc., of the mammal to be treated. The dosage can be conveniently administered, e.g., in divided doses up to four times a day or in sustained-release form.

As used herein, the term “treat,” “treatment,” or “treating,” refers to administering a therapeutic agent or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition. As used herein, a “therapeutic effect” relieves, to some extent, one or more of the symptoms of a disease or disorder. For example, a therapeutic effect may be observed by a reduction of the subjective discomfort that is communicated by a subject (e.g., reduced discomfort noted in self-administered patient questionnaire).

As used herein, the term “prophylaxis,” “prevent,” “preventing,” “prevention,” and grammatical variations thereof as used herein refers the preventive treatment of a subclinical disease-state in a subject, e.g., a mammal (including a human), for reducing the probability of the occurrence of a clinical disease-state. The method can partially or completely delay or preclude the onset or recurrence of a disorder or condition and/or one or more of its attendant symptoms or barring a subject from acquiring or reacquiring a disorder or condition or reducing a subject's risk of acquiring or requiring a disorder or condition or one or more of its attendant symptoms. The subject is selected for preventative therapy based on factors that are known to increase risk of suffering a clinical disease state compared to the general population. “Prophylaxis” therapies can be divided into (a) primary prevention and (b) secondary prevention. Primary prevention is defined as treatment in a subject that has not yet presented with a clinical disease state, whereas secondary prevention is defined as preventing a second occurrence of the same or similar clinical disease state.

As used herein, each of the terms “partial response,” “partial remission” and “PR” refers to the amelioration of a cancerous state, as measured by, for example, tumor size and/or cancer marker levels, in response to a treatment. In some embodiments, a “partial response” means that a tumor or tumor-indicating blood marker has decreased in size or level by about 50% in response to a treatment. The treatment can be any treatment directed against cancer, including but not limited to, chemotherapy, radiation therapy, hormone therapy, surgery, cell or bone marrow transplantation, and immunotherapy. The size of a tumor can be detected by clinical or by radiological means. Tumor-indicating markers can be detected by means well known to those of skill, e.g., ELISA or other antibody-based tests. A partial response of the target lesion can refer to at least a 30% decrease in the sum of the diameters of target lesions, taking as reference the baseline sum diameters.

As used herein, each of the terms “complete response,” “complete remission” or “CR” means that a cancerous state, as measured by, for example, tumor size and/or cancer marker levels, has disappeared following a treatment, including but are not limited to, chemotherapy, radiation therapy, hormone therapy, surgery, cell or bone marrow transplantation, and immunotherapy. The presence of a tumor can be detected by clinical or by radiological means. Tumor-indicating markers can be detected by means well known to those of skill, e.g., ELISA or other antibody-based tests. However, a “complete response” does not necessarily indicate that the cancer has been cured. A complete response may be followed by a relapse. A complete response of a target lesion includes disappearance of all target lesions and any pathological lymph nodes (whether target or non-target) having reduction in short axis to <10 mm. A complete response of a non-target lesion includes disappearance of all non-target lesions and normalization of tumor marker level (all lymph nodes must be non-pathological in size (<10 mm short axis)). If tumor markers are initially above the upper normal limit, they need to normalize for a patient to be considered in complete clinical response of a nontarget lesion. The duration of overall CR is measured from the time measurement criteria are first met for CR until the first date that progressive disease is objectively documented, or death due to any cause. Participants without events reported are censored at the last disease evaluation.

As used herein, the term “stable disease” or “SD” means neither sufficient shrinkage to qualify for PR nor sufficient increase to qualify for progressive disease (PD), taking as reference the smallest sum diameters while on study. Duration of stable disease is measured from the start of the treatment until the criteria for progression are met, taking as reference the smallest measurements recorded since the treatment started, including the baseline measurements.

As used herein, the term “progressive disease” or “PD” when refers to a target lesion means at least a 20% increase in the sum of the diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progressions). When progressive disease or PD refers to a non-target lesion, it means the appearance of one or more new lesions and/or unequivocal progression of existing non-target lesions. Unequivocal progression should not normally trump target lesion status. It must be representative of overall disease status change, not a single lesion increase.

As used herein, the term “best overall response” means the best response recorded from the start of the treatment until disease progression/recurrence (taking as reference for progressive disease the smallest measurements recorded since the treatment started). The patient's best response assignment depends on the achievement of both measurement and confirmation criteria. The duration of an overall response is measured from the time measurement criteria are met for CR or PR (whichever is first recorded) until the first date that recurrent or progressive disease is objectively documented (taking as reference for progressive disease the smallest measurements recorded since the treatment started, or death due to any cause. Participants without events reported are censored at the last disease evaluation).

As used herein, the term “MTD” means maximum tolerated dose.

As used herein, the term “DLT rate” means dose-limiting toxicity rate.

As used herein, the term “IC₅₀” means inhibitory drug concentration that produces 50% of the maximal effect.

As used herein, the term “SEM” means standard error of mean.

As used herein, the term “AUC(x-y)” means area under the curve, wherein “x” is the starting time in hours and “y” is the ending time in hours.

As used herein, the term “C_{avg}” means average concentration. As used herein, the term “Cₘₐₓ” means maximum concentration.

As used herein, the term “QD” means once daily.

As used herein, the term “ANC” means absolute neutrophil count.

As used herein, the term “EOT” means end-of-treatment.

As used herein, the term “ALT” means alanine aminotransferase.

As used herein, the term “AST” means aspartate aminotransferase.

As used herein, the term “CBC” means complete blood count.

As used herein, the term “CT” means computed tomography.

As used herein, the term “ctDNA” means circulating tumor DNA.

As used herein, the term “ECOG” means Eastern Cooperative Oncology Group (performance score).

As used herein, the term “ECG” means electrocardiogram.

As used herein, the term “MRI” means magnetic resonance imaging.

As used herein, the term “PK” means pharmacokinetic.

As used herein, the term “TNM” means tumor, lymph nodes, metastasis.

As used herein, the term “PBMC” means peripheral blood mononuclear cells.

As used herein, the term “CNS” means central nervous system.

As used herein, the term “concomitant medication” means medications (or treatments), other than the drugs used in a study, are taken or received by the patient during the course of the study (after the administration of the first dose of study drug and before the final study visit assessment).

As used herein, the term “tolerable” means a dose level where <1/6 participants have experienced a DLT, or the dose level that is declared the RP2D.

As used herein, the term “adverse event” or “AE” means an untoward medical occurrence in a subject administered a medicinal product that does not necessarily have a causal relationship with this treatment. An AE can be an unfavorable and unintended sign (including an abnormal laboratory finding), symptom, or disease temporally associated with the use of an investigational product, whether or not related to the investigational medicinal product. An adverse event may include worsening or exacerbation of the disease under study; worsening or exacerbation of pre-existing conditions or events; intercurrent illnesses; or drug interactions. Anticipated fluctuations of pre-existing conditions that do not represent a clinically significant exacerbation or worsening are not considered AEs. Surgical procedures are not adverse events; they are therapeutic measures for conditions that require surgery. The condition, provided it develops or is a worsening of a pre-existing condition for which the surgery is required, is the AE. Disease progression is an efficacy endpoint and is not an AE. A clinical event in the setting of disease progression would be considered an AE if it could not be unequivocally attributed to or consistent with expected disease progression.

As used herein, the term “expected adverse event” means an adverse event that are listed or characterized in the current adverse event list, the Package Insert (P.I.), the Investigator Brochure (IB.) or is included in the informed consent document as a potential risk.

As used herein, the term “unexpected adverse event” means an adverse event that is not listed in the P.I. or current I.B. or not identified. This includes adverse events for which the specificity or severity is not consistent with the description in the P.I. or I.B. For example, under this definition, hepatic necrosis would be unexpected.

As used herein, the term “severe adverse event” or “SAE” means an AE that (1) results in death (i.e., the AE actually causes or leads to death); (2) is life threatening (i.e., the AE, in the view of the investigator, places the subject at immediate risk of death, but does not include an AE that, had it occurred in a more severe form, might have caused death); (3) requires or prolongs inpatient hospitalization; (4) results in persistent or significant disability/incapacity (i.e., the AE results in substantial disruption of the subject's ability to conduct normal life functions); or (5) results in a congenital anomaly/birth defect in a neonate/infant born to a mother exposed to the IMP.

As used herein, the term “definite AE” means the AE is clearly related to the study treatment.

As used herein, the term “probable AE” means the AE is likely related to the study treatment.

As used herein, the term “possible AE” means the AE may be related to the study treatment.

As used herein, the term “unlikely AE” means the AE is doubtfully related to the study treatment.

As used herein, the term “unrelated AE” means the AE is clearly not related to the study treatment.

As used herein, the term “expected disease progression” means an event that is unequivocally related to disease progression, and that the clinical course is consistent with what would be expected for the patient's disease.

As used herein, the term “measurable lesion” means a lesion that can be accurately measured in at least one dimension (longest diameter to be recorded) as ≥20 mm by chest x-ray or ≥10 mm with CT scan, MRI, or calipers by clinical exam. Tumor lesions that are situated in a previously irradiated area might or might not be considered measurable. Cystic lesion thought to represent cystic metastases are measurable lesions if they meet the definition of measurability described above. However, they are target lesions if non-cystic lesions are also present in the same participant. Clinical lesions are measurable when they are superficial (e.g., skin nodules and palpable lymph nodes) and ≥10 mm in diameter as assessed using calipers (e.g., skin nodules).

As used herein, the term “malignant lymph node” means a pathologically enlarged and measurable lymph node with ≥15 mm in short axis when assessed by CT scan.

As used herein, the term “non-measurable disease” means a small lesion (or a site of disease) where the longest diameter <10 mm or pathological lymph nodes with ≥10 to <15 mm short axis. Bone lesions, leptomeningeal disease, ascites, pleural/pericardial effusions, lymphangitis cutis/pulmonitis, inflammatory breast disease, abdominal masses (not followed by CT or MRI), and cystic lesions are examples of non-measurable disease. Cystic lesions that meet the criteria for radiographically defined simple cysts are not malignant lesions (neither measurable nor non-measurable) and are simple cysts.

As used herein, the term “target lesion” means all measurable lesions up to a maximum of 2 lesions per organ and 5 lesions in total, which is representative of all involved organs. Target lesions are selected on the basis of their size (lesions with the longest diameter), representative of all involved organs, but in addition should be those lending themselves to reproducible repeated measurements. When the largest lesion does not lend itself to reproducible measurement, the next largest lesion that can be measured reproducibly is the target lesion.

As used herein, the term “non-target lesions” means all lesions (or sites of disease) that are not target lesions. Non-target lesions include any measurable lesions over and above the 5 target lesions.

As used herein, the term “overall survival” or “OS” means the time from randomization (or registration) to death due to any cause. Participants survived are censored on date last known alive.

As used herein, the term “progression-free survival” or “PFS” means the time from randomization (or registration) to the earlier of progression or death due to any cause. Participants alive without disease progression are censored on date of last disease evaluation.

As used herein, the term “time to progression” or “TTP” means the time from randomization (or registration) to progression. Participants without progression reported are censored on date of last disease evaluation.

As used herein, the term “CXD1” means Day 1 of Cycle X. For example, C1D1 means Day 1 of Cycle 1, which is before the subject receiving the intended treatment. C2D1 means Day 1 of Cycle 2.

### Cancer

Methods, compositions and kits disclosed herein can be used for treating cancer and/or tumor. In some embodiments, a method for treating cancer and/or tumor comprises administrating an MTDP inhibitor (e.g., paclitaxel), or a pharmaceutically acceptable salt, solvate, stereoisomer thereof, and a PLK1 inhibitor (e.g., onvansertib), or a pharmaceutically acceptable salt, solvate, stereoisomer thereof, to a subject (e.g., a patient) in need thereof.

The methods, compositions and kits disclosed herein can be used to treat various types of cancer and/or tumor. The cancer and/or tumor can be a solid tumor, a liquid tumor, or a combination thereof. In some embodiments, the cancer and/or tumor is a hematologic or solid cancers. The cancer can be breast cancer, pancreatic cancer, gastric cancer, gastroesophageal cancer, esophageal cancer, lung cancer, prostate cancer, cervical cancer, colorectal cancer, thyroid cancer, bladder cancer, head and neck cancer, brain and central nervous system cancer, liver cancer, gallbladder cancer, cholangiocarcinoma, ovarian cancer, vaginal cancer, renal cancer, endometrial cancer, skin cancer, testicular cancer, thymus cancer, unspecified cancer, adenocarcinoma, leukemia, lymphoma, sarcoma, other neoplastic malignancies and/or a combination thereof. The breast cancer can be invasive breast cancer, unresectable breast carcinoma, locally advanced breast cancer, metastatic breast cancer, inflammatory breast cancer, triple negative breast cancer (TNBC), hormone receptor/growth factor receptor-negative breast cancer, HER2-negative breast cancer, and/or hormone receptor negative breast carcinoma. The pancreatic cancer can be metastatic pancreatic ductal adenocarcinoma, pancreatic ductal adenocarcinoma, and/or borderline resectable pancreatic adenocarcinoma. The lunch cancer can be lung adenocarcinoma and/or mesothelioma. The prostate cancer can be metastatic castration-resistant prostate cancer, and/or prostate adenocarcinoma. The colorectal cancer can be metastatic colorectal cancer and/or colon cancer. The bladder cancer can be urothelial cancer. The head and neck cancer can be squamous cell carcinoma of the head and neck, locally advanced or metastatic cancer of the head and neck, mouth cancer, pharynx cancer, larynx cancer, recurrent squamous cell carcinoma of the larynx, salivary gland cancer, hypopharynx cancer, squamous cell carcinoma of the lip and oral cavity, tongue cancer, nasopharynx cancer, and/or oropharynx cancer. The brain and central nervous system cancer can be glioblastoma, glioma, and/or neuroblastoma. The liver cancer can be intrahepatic cholangiocarcinoma (iCCA) and/or hepatocellular carcinoma. The ovarian cancer can be fallopian tube cancer, peritoneal cancer. The renal cancer can be renal cell carcinoma, advanced or metastatic renal cell cancer, and/or metastatic transitional cell cancer of the renal pelvis and ureter. The skin cancer can be melanoma, squamous cell carcinoma and/or metastatic Merkel cell carcinoma. The leukemia can be acute myeloid leukemia and/or multiple myeloma. The lymphoma can be B cell lymphoma, T cell lymphoma, Non-Hodgkin's lymphoma, and/or follicular lymphoma. The sarcoma can be rhabdomyosarcoma. In some embodiments, the cancer and/or tumor is hematologic or solid cancers. The hematologic or solid cancers is not a gastric cancer or a lung cancer. The hematologic or solid cancers include breast cancer (e.g., invasive breast cancer, unresectable breast carcinoma, locally advanced breast cancer, metastatic breast cancer, inflammatory breast cancer, TNBC, hormone receptor/growth factor receptor-negative breast cancer, HER2-negative breast cancer, and/or hormone receptor negative breast carcinoma); pancreatic cancer (e.g., metastatic pancreatic ductal adenocarcinoma, pancreatic ductal adenocarcinoma, and/or borderline resectable pancreatic adenocarcinoma); gastroesophageal cancer; esophageal cancer; prostate cancer (e.g., metastatic castration-resistant prostate cancer and/or prostate adenocarcinoma); cervical cancer; colorectal cancer (e.g., metastatic colorectal cancer and/or colon cancer); thyroid cancer; bladder cancer (e.g., urothelial cancer); head and neck cancer (e.g., squamous cell carcinoma of the head and neck, locally advanced or metastatic cancer of the head and neck, mouth cancer, pharynx cancer, larynx cancer, recurrent squamous cell carcinoma of the larynx, salivary gland cancer, hypopharynx cancer, squamous cell carcinoma of the lip and oral cavity, tongue cancer, nasopharynx cancer, and/or oropharynx cancer); brain and central nervous system cancer (e.g., glioblastoma, glioma, and/or neuroblastoma); liver cancer (e.g., intrahepatic cholangiocarcinoma (iCCA), and/or hepatocellular carcinoma); gallbladder cancer; cholangiocarcinoma; ovarian cancer; vaginal cancer; renal cancer (e.g., renal cell carcinoma, advanced or metastatic renal cell cancer, and/or metastatic transitional cell cancer of the renal pelvis and ureter); endometrial cancer; skin cancer (e.g., melanoma, squamous cell carcinoma, and/or metastatic Merkel cell carcinoma); testicular cancer; thymus cancer; unspecified cancer; adenocarcinoma; leukemia (e.g., acute myeloid leukemia); lymphoma; sarcoma (e.g., rhabdomyosarcoma); other neoplastic malignancies; and/or a combination thereof. In some embodiments, the cancer and/or tumor is a solid tumor, including but are not limited to, melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, Merkel cell carcinoma, brain and central nervous system cancers, and any combination thereof. In some embodiments, the cancer is a liquid tumor. In some embodiments, the cancer is a hematological cancer. Non-limiting examples of hematological cancer include T cell lymphoma, B cell lymphoma, Non-Hodgkin's lymphoma (“NHL”), Follicular lymphoma (“FL”), acute myeloid leukemia (“AML”), and multiple myeloma (“MM”). In some embodiments, the disease or condition provided herein includes refractory or recurrent malignancies, whose growth may be inhibited using the methods and compositions disclosed herein.

In some embodiments, the cancer and/or tumor is leukemia (e.g., AML); NHL; metastatic CRC; metastatic castration resistant prostate cancer (mCRPC); pancreatic cancer (e.g., metastatic castration-resistant prostate cancer); adrenocortical carcinoma (ACC); breast cancer (e.g., TNBC); colorectal cancer (e.g., metastatic colorectal cancer with KRAS mutations); small cell lung cancer (SCLC); ovarian cancer; and/or a combination thereof.

In some embodiments, the cancer is an invasive cancer unresectable locally advanced or metastatic disease, for example an invasive breast cancer. In some embodiments, the cancer is an inflammatory cancer, for example breast cancer, including TNBC. In some embodiment, the cancer has a histological or cytological profile with ER≤10%, PR≤10%, Her-2-neu negative per ASCO/CAP 2018 guidelines (0-1+ by immuno-histochemistry (IHC) or fluorescence in situ hybridization (FISH)-negative).

The cancer and/or tumor can be a cancer and/or tumor having abnormal alterations to PLK1 gene or protein. For example, the abnormal alterations can include one or more PLK1 alterations and/or PLK1 aberrant activation such as copy number alteration (CNA), single-nucleotide variation (SNV), and gene rearrangement or fusions. Non-limiting exemplary cancer and/or tumor with PLK1 alterations include cancer with PLK1 gene or protein amplification, PLK1 gene or protein modification, PLK1 gene deletion, PLK1 gene or protein overexpression, elevated PLK1 gene or protein expression, and/or a combination thereof. In some embodiments, the cancer and/or tumor can be a PLK1-amplified cancer, in which PLK1 gene and/or protein is amplified, for example, as a result of gene duplication and/or aberrant gene transcriptional control. For example, the cancer with PLK1 amplification can be a cancer with higher PLK1 mRNA and/or protein levels as compared to healthy tissues. In some embodiments, a kidney, lung, breast, colon, skin, and/or head and neck can exhibit a high PLK1 gene or protein amplification, and the resulting cancer and/or tumor is suitable for treatment by the methods and compositions disclosed herein. In heterogenous cancer types, the cancer/or tumor can include a subtype that has an abnormally high expression of PLK1 gene and/or protein. Non-limiting examples of cancers and/or tumors with subtypes having high PLK1 gene and/or protein expression include lymphoma (e.g., B-cell lymphoma, diffuse large B-cell lymphoma); testicular cancer (e.g., testicular germ cell cancer); cervical cancer; head and neck cancer (e.g., uveal melanoma, adenoid cystic carcinoma); ovarian cancer, uterine cancer (e.g., uterine carcinosarcoma); colorectal cancer; thyroid cancer (e.g., thymoma); bladder cancer; lung cancer (lung squamous, lung adeno, mesothelioma); uterine cancer; skin cancer (e.g., melanoma); sarcoma; brain tumor (e.g., glioblastoma, glioma); leukemia (e.g., acute myeloid leukemia); breast cancer; pancreatic cancer; bile duct cancer (e.g., cholangiocarcioma); liver cancer; renal cancer (e.g., renal cell carcinoma, clear cell renal cell carcinoma, chromophobe renal cell carcinoma, papillary renal cell carcinoma); neuroendocrine tumor (e.g., pheochromocytoma/paraganglioma). The cancer and/or tumor with amplified PLK1 can be node-positive tumors, aggressive tumors and/or invasive tumors. The cancer and/or tumor with amplified PLK1 can have a shorter disease-free survival as compared to cancer and/or tumor with normal levels of PLK1. The cancer and/or tumor can exhibit a high relapse and/or resistance to traditional and/or mono-therapies, such as chemotherapy and/or radiotherapy. The prostate cancer can be resistant to mono-treatment with a MTDP inhibitor or a MTDP inhibitor.

The cancer and/or tumor can be a breast cancer and/or breast tumor. In some embodiments, the breast cancer and/or breast tumor is unresectable locally. In some embodiments, the breast cancer and/or breast tumor is advanced and/or metastatic. In some embodiments, the breast cancer and/or breast tumor has cells with unstable genome. In some embodiments, the breast cancer and/or breast tumor is a basal-like subtype, characterized by overexpression of epidermal growth factor receptor (EGFR), loss of phosphatase and tensin homolog (PTEN), mutations in the TP53 gene (mutp53), and exhibiting high overall genomic instability among the subtypes. In some embodiments, the breast cancer and/or breast tumor has low pathologic complete response rate following neoadjuvant chemotherapy. In some embodiments, the breast cancer and/or breast tumor are intrinsically resistant to chemotherapy and/or have a poor overall survival rate. In some embodiments, the breast cancer and/or tumor is invasive breast cancer, unresectable breast carcinoma, locally advanced breast cancer, metastatic breast cancer, inflammatory breast cancer, TNBC, hormone receptor/growth factor Receptor-negative breast cancer, HER2-negative breast cancer, and/or hormone receptor negative breast carcinoma. In some embodiments, the breast cancer is invasive breast cancer, unresectable breast carcinoma, locally advanced breast cancer, metastatic breast cancer, inflammatory breast cancer, TNBC, hormone receptor/growth factor receptor-negative breast cancer, HER2-negative breast cancer, hormone receptor negative breast carcinoma, and/or a combination thereof. In some embodiments, the breast cancer and/or breast tumor is a biologically aggressive form of breast cancer. In some embodiments, the breast cancer and/or breast tumor lacks an estrogen receptor (ER), lacks a progesterone receptor, and lacks human epidermal growth factor receptor 2 (HER2) gene amplification. In some embodiments, the breast cancer and/or breast tumor is associated with a high mortality rate, with a median survival of less than two years from the time of metastasis. In some embodiments, the breast cancer and/or breast tumor is TNBC. Novel target therapies and target combination therapies are needed for TNBC, which has such a poor prognosis and short overall survival. Targeted therapy for TNBC, for example, can involve immune checkpoint inhibitors (CPIs). With more than 60% of metastatic TNBC are negative for PD-L1 expression (combined positive score (CPS)<10), in some cases, the therapy can target programmed death 1 (PD-1)/PD-L1. In some cases, paclitaxel alone is a standard first line therapy. In a KEYNOTE-355 study, a regimen of chemotherapy (either with a taxane or carboplatin plus gemcitabine) and Pembrolizumab (a monoclonal antibody directed against the PD-1 receptor) significantly (27%) reduced the risk of death (CPS>10) for patients with metastatic TNBC whose tumors were strongly positive for PD-L1, as compared to chemotherapy vs placebo. Recently, the FDA approved Sacituzumab govitecan, an antibody drug conjugate directed against (tumor-associated calcium signal transducer 2 (TROP2), as a 2nd and 3rd line therapy for metastatic TNBC. However, existing therapies are not ideal. Novel target therapies and target combination therapies are needed for basal-like breast cancer, which are intrinsically resistant to chemotherapy and patients with this type of breast cancer have a poor overall survival rate. Among the heterogenous TNBC, basal-like subtype, identified through gene expression analysis, is characterized by overexpression of EGFR, and PTEN, and mutations in TP53 gene, exhibiting the greatest overall genomic instability among the subtypes. Patients with basal-like breast cancer have low pathological complete response rate following neoadjuvant chemotherapy. It has been identified that PLK1 is an important gene for growth and survival of breast cancer cells with unstable genome, including TNBC.

Paclitaxel is an effective agent in the treatment of metastatic breast cancer. Weekly paclitaxel has been demonstrated to have better activity and less myelosuppression compared to the every-3 weeks schedule. Neuropathy, when present, is usually of mild or moderate severity and generally reversible. In a large phase 2 trial of paclitaxel 80 mg/m² weekly for 4 weeks per 4-week cycle including 212 metastatic breast cancer patients, therapy was generally well tolerated. Grade 3 or 4 neutropenia occurred in 31 patients (15%). All but two patients who developed grade 3 or 4 neutropenia had received prior chemotherapy, and five of these patients had received prior high-dose chemotherapy. Grade 3 anemia was encountered in 18 patients (9%). Grades 3 and 4 thrombocytopenia were experienced by one patient, respectively. There were 30 patients (14%) enrolled with preexisting grade 1 neuropathy. Two of these patients eventually developed grade 3 neuropathy after 5 and 11 courses of therapy. Overall, the incidence of any grade of neuropathy was 69%. However, grade 3 neuropathy was encountered in only 20 patients (9%), and no patient experienced grade 4 neuropathy. The median number of courses of therapy until development of grade 2 or 3 neuropathy, including patients with preexisting grade 1 toxicity, was five courses (20 weeks) for either and ranged from 1 to 13 courses. Among the 177 assessable patients, overall response rate was 21.5% (95% confidence interval, 15.4% to 27.5%). Responses occurred in 23 (17.6%) of the 131 assessable patients who had received prior anthracycline therapy and in 7 (15.6%) of the 45 assessable patients who had received prior taxane therapy. The median time to progression for assessable patients was 142 days (4.7 months). Median times to progression for patients who had received no prior chemotherapy for metastatic disease, one prior regimen, and two prior regimens were 174 days (5.7 months), 140 days (4.6 months), and 85 days (2.7 months), respectively.

### MTDP Inhibitors and PLK Inhibitors

Methods, compositions and kits disclosed herein can be used for treating cancer and/or tumor, for example breast cancer; pancreatic cancer; gastric cancer; gastroesophageal cancer; esophageal cancer; lung cancer; prostate cancer; cervical cancer; colorectal cancer; thyroid cancer; bladder cancer; head and neck cancer; brain and central nervous system cancer; liver cancer; gallbladder cancer; cholangiocarcinoma; ovarian cancer; vaginal cancer; colorectal cancer; renal cancer; endometrial cancer; skin cancer; testicular cancer; thymus cancer; unspecified cancer; adenocarcinoma; leukemia; lymphoma; sarcoma; other neoplastic malignancies; and/or a combination thereof. In some embodiments, a method for treating cancer and/or tumor comprises administrating α tubulin targeting agent, such as a MTDP inhibitor (e.g., paclitaxel), or a pharmaceutically acceptable salt, solvate, stereoisomer thereof, and a PLK1 inhibitor (e.g., onvansertib), or a pharmaceutically acceptable salt, solvate, stereoisomer thereof, to a subject (e.g., a patient) in need thereof. The method can comprise administering a pharmaceutically effective amount of the MTDP inhibitor (e.g., paclitaxel) and a pharmaceutically effective amount of the PLK1 inhibitor (e.g., onvansertib).

### MTDP Inhibitors

Microtubules are highly dynamic polymers of tubulins that makes up the cytoskeleton are crucial for cell shape, intracellular trafficking, cell division and cancer. Microtubule targeting agents, such as compounds that suppresses microtubule dynamics (including spindle-microtubule dynamics), microtubule depolymerization inhibitors, and microtubule polymerization inhibitors, is an important group of antimitotic compounds with anticancer properties and can be used as a first-line cancer therapeutic. A suppression of microtubule dynamics, and in particular, a suppression of the spindle-microtubule dynamics, can prevent a cell from completing mitosis. A cell that is blocked in a prometaphase/metaphase-liked state can eventually undergo apoptosis.

Tubulin targeting agents can be used as a first-line cancer therapeutic. In general, tubulin targeting agents interfere with microtubule dynamics by stabilizing or destabilizing microtubules, making they a suitable first-line cancer therapeutic. However, tubulin targeting agents also suffer from tissue specificity, innate and/or acquired drug resistance, and systemic toxicity. Moreover, some tubulin targeting agent induced changes in microtubule dynamics can be detrimental to cancer treatment. For example, tubulin targeting agents can alter microtubule chromosome attachment and chromosome missegregation causing chromosome instability and aneuploidy. In some cases, tubulin targeting agents can drive tumorigenesis, cancer development, drug resistance, therapy failure, metastasis, poor prognosis, polyploid cell formation (including polyploid giant cells), migratory capabilities, and invasive phenotypes.

Binding to the taxane site, located at the β tubulin monomer in the lumen of microtubules, can stabilize the microtubule lattice. For example, binding of paclitaxel or epothilone to the taxane site can stabilize the microtubule lattice, albeit by different mechanisms. Other examples of agents that bind to the taxane site includes PM060184 (plocabulin) and the covalent tubulin inhibitor, zampanolide. Binding to the laulimalide or peloruside site, located at a β tubulin pocket facing the outside of the microtubule, inhibits microtubule disassembly. For example, binding of laulimalide and peloruside to this site clamps protofilaments and prevents microtubule disassembly. Binding to the Vinca domain, located at the inter-dimer interface between two longitudinally aligned tubulin dimers, can inhibit tubulin polymerization. For example, binding of vincristine to the Vinca domain can inhibit tubulin assembly by sequestering tubulin into paracrystalline aggregates. Binding to the colchicine site, located in a deep pocket between the tubulin dimer, inhibits microtubule formation. For example, binding of colchicine, benzimidazoles (e.g., nocodazole), or combretastatins to the colchicine site can inhibit microtubule polymerization by preventing the conformation changes in tubulin necessary for polymerization. Binding to the maytansine domain, located on an exposed β tubulin pocket proximal to the Vinca site, inhibits polymerization at the plus end. For example, binding of maytansine and spongistatin to the maytansine domain can inhibit new tubulins from being added to the plus end. Binding to the pironetin site, located on the α tubulin, destabilizes microtubules. For example, binding of pironetin to this site can inhibit logitudinal tubulin interactions and prevent heterodimer formation. Binding to the gatorbulin site, located on the α tubulin proximal to the colchicine site, inhibits tubulin polymerization. For example, binding of Cevipabulin to the gatorbulin site can create wedge through two longitudinally aligned tubulin dimers at the tip of the microtubules.

MTDP inhibitors include, but are not limited to, diterpenoid; taxanes; taxane-derived diterpenoids; taxoids; and the derivatives, analogues, precursors, and conjugates thereof. In some embodiments, the MTDP inhibitor is paclitaxel, docetaxel, acetyltaxol, paclitaxel; lutetium Lu 177 vipivotide tetraxetan; 7-hexanoyltaxol; cabazitaxel; larotaxel; milataxel; ortataxel; tesetaxel; taxoprexin; opaxio; taxoprexin (DHA-paclitaxel); Poly(L-glutamic acid)-paclitaxel; abraxane; SB-T-1214; SB-T1216; SB-T121602; SB-T-12854; DHA-SB-T1214; abeo-taxanes. The abeo-taxanes can be abeo-taxane 15a.2; docetaxl-d9-t-Boc; docetaxel-f3-t-Boc; cabazitaxel-7,10-d6; Poly(glutamyl-glutamate)-taxane and/or derivatives, analogues, precursors, and conjugates thereof.

Non-limiting examples of microtubule targeting agents include compounds that target the taxane site of the microtubules (e.g., paclitaxel); compounds that target the Vinca domain of the microtubules (e.g., vinflunine); compounds that target the colchicine domain (e.g., cyclohexanediones with a distal 2-substituted benzofurane, heterocyclic nitrogen compound such as Plinabulin, verubulin, and ABT-751, combretastatin such as ombrabulin and fosbretabulin); and compounds that target other microtubule binding sites (e.g., estramustine). Microtubule targeting agents also include other microtubule-destabilizing agents, such as anti-tussive noscapine; maytansine and/or maytansine/auristatin conjugate (e.g., TDM1, brentuximab vedotin, and SAR33419); rhizoxin; spongistatins; podophyllotoxin; steganacins; curacins; anti-mitotic herbicides that inhibit microtubule polymerization; antifungal and antihelmintic agents; and certain psychoactive drugs (e.g., dilantin, vinblastine, chlorpromazine). Microtubule targeting agents also include other microtubule-stabilizing agents, such as the eleutherobins, sarcodictyins, laulimalide, rhazinalam, steroids, and polyisoprenyl benzophenones.

Many microtubule-targeted compounds are tissue specific. For example, it has been suggested that paclitaxel is very effective against ovarian tumors, breast tumors, and lung tumors, but has little efficacy against many other solid tumors, such as kidney carcinoma, colon carcinoma, and some sarcomas. As another example, it was found that Vinca alkaloids are frequently most effective against hematological cancers, but often ineffective against many solid tumors.

Drug resistance of microtubule-targeted compounds is a complex and largely unsolved problem. In some cases, drug resistance is correlated with overexpression of ATP-dependent drug efflux pumps or ATP-binding cassettes, a class of membrane transporter proteins. The membrane pumps remove the drug at the intracellular level and can lead to resistance to drugs having different chemical structures (e.g., both paclitaxel and Vinca alkaloids). Tissue specific differences in regulatory factors (e.g., differences in regulatory protein expression, post-translational modification of tubulin, and expression of different tubulin isotype) can contribute to tissue specific sensitivity and/or resistance to microtubule-targeted compounds.

In some embodiments, compounds that target the taxane site of the microtubules include paclitaxel, TL00139, and paclitaxel analogues, docetaxel (taxotere), epothilone (e.g., BMS-247550, epothilones B and D), ixabepilone, discodermolide, and other similar depolymerization inhibitors. In some embodiments, compounds that target near the taxane site of microtubule include sarcodictyins and eleutherobin.

MTDP inhibitors that bind to the taxane site of tubulin, unlike Vinca alkaloids, stimulate microtubule polymerization and are a group of important compounds to treat, for example, breast cancer, ovarian cancer, non-small-cell lung cancer, and Kaposi's sarcoma. However, side effects include neurotoxicity and myelosuppression. The MTDP inhibitors that target the taxane site in the β-subunit bind to the surface of a polymerized microtubule. This stabilizes the microtubule and increases microtubule polymerization and increases its affinity for neighboring tubulin molecules. In addition, at low paclitaxel concentrations, polymerization does not increase but microtubule dynamics can be fully stabilized. For example, the binding of a small number of paclitaxel molecules (e.g., one paclitaxel per several hundred tubulin molecules) can stabilize the dynamics of the microtubules (e.g., reduce the rate or extent of microtubule shortening by approximately 50%). In HeLa cells, about 8 nM to about 10 nM of paclitaxel can half-maximally block mitosis without increasing microtubule-polymer mass. In general, inhibitor mediated suppression of microtubule dynamics ultimately blocks mitosis and leads to apoptosis.

In some embodiments, the MTDP inhibitor is paclitaxel, a paclitaxel derivative or analog (e.g., docetaxel), or a pharmaceutically acceptable salt thereof. Paclitaxel, also known as 5β,20-Epoxy-1,2α,4,7β,10β,13α-hexahydroxytax-11-en-9-one 4,10-diacetate 2-benzoate 13-ester with (2R,3S)-N-benzoyl-3-phenylisoserine, has the empirical formula C₄₇H₅₁NO₁₄ and a molecular weight of 853.9, and is a tricyclic diterpenoid with a taxane ring (Formula 1). The paclitaxel structures essential for antitumor activity include the taxane ring, the C13 side chain, the oxetane ring, the 2′ position of the hydroxy group, and the homochiral ester chain. The paclitaxel structures not essential for antitumor activity include the hydroxyl group at C7 and acetylation of the C10 hydroxyl group.

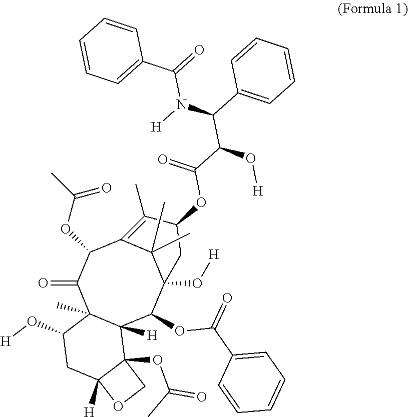 [SMILES: CC(=O)O[C@H]1C(=O)[C@@]2(C)[C@H]([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O]

Paclitaxel is generally obtained via a semi-synthetic process from Taxus baccata, is highly lipophilic, is insoluble in water, and melts at around 216° C.-217° C. In some embodiments, the paclitaxel is administered by intravenous injection. In some embodiments, the paclitaxel is administered orally. In some embodiments, the paclitaxel is combined with additives. For example, paclitaxel can be formulated with a surfactant, including heterogeneous non-ionic surfactants such as Cremophor EL (a polyoxyethylated castor oil) and/or dehydrated alcohol.

Paclitaxel can be formulated as a nanomedicine. In some embodiments, the paclitaxel is bound to albumin, to form a 130 nm nanomedicine (e.g., nab-PTX). In some embodiments, the paclitaxel is formulated in a polymeric micelle such as with mPEG-PDLLA to form a 25 nm nanomedicine, with PVP-bPNIPAAM to form an 80 nm-100 nm nanomedicine, with N-tr-Lc methyl ester and N.13cr-Lc methyl ester to form a 20 nm to 60 nm nanomedicine. In some embodiments, the paclitaxel is formulated in a liposome such as with lecithin and cholesterol to form a 400 nm nanomedicine. In some embodiments, the paclitaxel is formulated in polymeric lipid-nanoparticles such as with polyvinyl-pyrrolidone, cholesteryl sulfate, and caprylic to form a 100 nm nanomedicine. In some embodiments, the paclitaxel is formulated in an emulsion with monoolein, tricaprylin, and Tween 80. The paclitaxel can be formulated in a dimer.

Paclitaxel can be administered orally, e.g., the paclitaxel can be conjugated to a chitosan, a lipid derivative, a nanocochleate, a hyaluronic acid-octadecylamine micelle, or an oil based nanocarrier. As another example, the paclitaxel can be loaded into a milk derived exosome.

Paclitaxel has been approved for use in, for example, ovarian cancer, breast cancer, non-small cell lung cancer, pancreatic cancer, bladder cancer, AIDS-related Kaposi's sarcoma, and gastric cancer. Paclitaxel is an effective agent in the treatment of metastatic breast cancer. Weekly paclitaxel has demonstrated to have better activity and less myelosuppression compared to the every-3 weeks schedule. Neuropathy, when present, is usually of mild or moderate severity and generally reversible. In a large phase 2 trial of paclitaxel 80 mg/m² weekly for 4 weeks per 4-week cycle including 212 metastatic breast cancer patients, therapy was generally well tolerated. Grade 3 or 4 neutropenia occurred in 31 patients (15%). All but two patients who developed grade 3 or 4 neutropenia had received prior chemotherapy, and five of these patients had received prior high-dose chemotherapy. Grade 3 anemia was encountered in 18 patients (9%), and grades 3 and 4 thrombocytopenia were experienced by one patient, respectively. There were 30 patients (14%) enrolled with preexisting grade 1 neuropathy. Two of these patients eventually developed grade 3 neuropathy after five and 11 courses of therapy. Overall, the incidence of any grade of neuropathy was 69%. However, grade 3 neuropathy was encountered in only 20 patients (9%), and no patient experienced grade 4 neuropathy. The median number of courses of therapy until development of grade 2 or 3 neuropathy, including patients with preexisting grade 1 toxicity, was five courses (20 weeks) for either and ranged from 1 to 13 courses. In the 177 assessable patients, overall response rate was 21.5% (95% confidence interval, 15.4% to 27.5%). Responses occurred in 23 (17.6%) of the 131 assessable patients who had received prior anthracycline therapy and in seven (15.6%) of the 45 assessable patients who had received prior taxane therapy. The median time to progression for assessable patients was 142 days (4.7 months). Median times to progression for patients who had received no prior chemotherapy for metastatic disease, one prior regimen, and two prior regimens were 174 days (5.7 months), 140 days (4.6 months), and 85 days (2.7 months), respectively. In some embodiments, paclitaxel is administrated to patients. Docetaxel has been used to treat breast cancer, lung cancer, and prostate cancer.

An MTDP inhibitor can be administered by any suitable routes, including but not limited to oral, topical (including buccal and sublingual), rectal, vaginal, transdermal, parenteral, subcutaneous, intraperitoneal, intrapulmonary, intradermal, intrathecal, epidural, and intranasal administration. Parenteral administration (e.g., injection) can include intramuscular, intravenous, intraarterial, intraperitoneal, or subcutaneous administration.

### PLK1 Inhibitors

Polo-like kinases (PLK) are a family of five highly conserved serine/threonine protein kinases. PLK1 is a master regulator of mitosis and is involved in several steps of the cell cycle, including mitosis entry, centrosome maturation, bipolar spindle formation, chromosome separation, and cytokinesis. It is also critical for the entry and progression through mitosis, regulates progression of cells through the G2 phase of the cell cycle by phosphorylating forkhead box protein M1 (FOXM1), which then regulates the expression of cyclins and other genes necessary for cells to progress through the cell cycle. PLK1 has been shown to be overexpressed in solid tumors and hematologic malignancies, including breast cancers. Overall survival of breast cancer patients with high PLK1 expression is lower than breast cancer patients with low PLK1 expression. PLK1 expression level was higher in TNBC as compared to adeno-cavity Type A, adeno-cavity Type B, and HER-2 overexpression type breast cancer. PLK1 inhibition induces G2-M-phase arrest with subsequent apoptosis of cancer cells, and has emerged as a promising targeted therapy. Several PLK inhibitors have been studied in clinical trials. In the early pre-clinical development of PLK1 targeted drugs, cancer cells with TP53 mutation (mutp53) were more responsive and had lower IC₅₀ than cell lines with wild type (wtp53), which are consistent with the lack of checkpoint control and genomic instability associated with mutp53. These observations confirm the importance of PLK1 function for progression through G2 and M phases of the cell cycle. The pyruvate dehydrogenases kinase 1 (PDK1), PLK1, and MYC have also been suggested to be important in driving the expression of a set of genes associated with cancer stem cell self-renewal. Thus, it is possible that blocking PLK1 function and affecting the ability of cancer cells with unstable genomes to progress through mitosis can increase the overall sensitivity of cells to taxanes, such as paclitaxel. PLK1 has been identified as a therapeutic target for TNBC through siRNA-mediated screen and inhibition of PLK1 by siRNA-mediated knockdown or a chemical inhibitor promoted cell cycle arrest and apoptosis in multiple TNBC lines. The lack of a druggable target is a reason for poor prognosis of TNBC. The effectiveness and unique action profile of PLK1 inhibition, in addition to the relatively specific expression of PLK1 in TNBC tissue, suggest that PLK1 is a promising molecular target for TNBC.

In a randomized phase II study of patients with AML who were treatment-naïve yet unsuitable for induction therapy, the pan-PLK inhibitor, volasertib (BI6727), administered intravenously in combination with low-dose Ara-C(cytarabine) (LDAC) showed a significant increase in OS when compared with LDAC alone. A subsequent randomized phase III study identified no benefit of the combination and described an increased risk of severe infections. PLK1 facilitates homologous recombination (HR) during Double Strand DNA Break (DSB) Repair. PLK1 phosphorylates Rad51 and BRCA1, facilitating their recruitment to DSB sites and thereby HR-mediated DNA repair.

Onvansertib (also known as PCM-075, NMS-1286937, NMS-937, “compound of formula (I)” in U.S. Pat. No. 8,927,530; IUPAC name 1-(2-hydroxyethyl)-8-{[5-(4-methylpiperazin-1-yl)-2-(trifluoromethoxy) phenyl]amino}-4,5-dihydro-1H-pyrazolo[4,3-h]quinazoline-3-carboxamide), or a pharmaceutically acceptable salt, is a selective ATP-competitive PLK1 inhibitor. Onvansertib can be formulated, for example, with an additive such as free base, lactose monohydrate, pregelatinized starch and glyceryl beneate. In some embodiments, the onvansertib is formulated for oral administration, such as in a hard gelatin capsule

Biochemical assays demonstrated high specificity of onvansertib for PLK1 among a panel of 296 kinases, including other PLK members. Onvansertib has potent in vitro and in vivo antitumor activity in models of both solid and hematologic malignancies. Onvansertib is the first PLK1 specific ATP competitive inhibitor administered by oral route to enter clinical trials with proven antitumor activity in different preclinical models. Onvansertib has shown a promising safety profile in a phase I clinical trial as single agent. In addition, clinical investigations of onvansertib includes onvansertib in combination with abiraterone and prednisone in adult patients with metastatic castration-resistant prostate cancer, onvansertib in combination with FOLFIRI and bevacizumab in adult patients with KRAS-mutated metastatic colorectal cancer, and onvansertib in combination with nanoliposomal irinotecan and 5-FU in patients with metastatic pancreatic cancer. As described herein, in the presence of genomic instability, onvansertib can synergize with paclitaxel, and thereafter achieve good anti-tumor activity at a lower dose compared to single agent, and without drug specific toxicity.

Onvansertib also inhibited cell proliferation at nanomolar concentrations in AML cell lines and tumor growth in xenograft models of AML. In addition, onvansertib significantly increased cytarabine antitumor activity in disseminated models of AML.

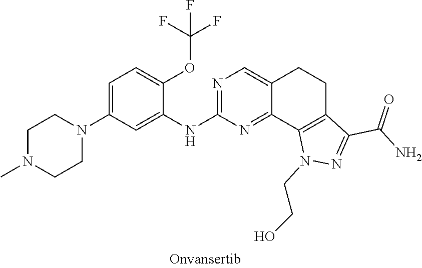 [SMILES: CN1CCN(c2ccc(OC(F)(F)F)c(Nc3ncc4c(n3)-c3c(c(C(N)=O)nn3CCO)CC4)c2)CC1]

Onvansertib shows high potency in proliferation assays having low nanomolar activity on a large number of cell lines, from both solid and hematologic tumors. Onvansertib has a relative short half-life of 24 h and is highly potent against the PLK1 enzyme ([IC50]=2 nM). In comparison, low or no activity was observed on a panel of 63 kinases other than PLK1 (IC50 >500 nM), including the PLK members PLK2 and PLK3 (IC50>10 μM). Onvansertib potently causes a mitotic cell-cycle arrest followed by apoptosis in cancer cell lines and inhibits xenograft tumor growth with a clear PLK1-related mechanism of action at well tolerated doses in mice after oral administration. In addition, onvansertib showed activity in combination therapy with approved cytotoxic drugs, such as irinotecan, in which there was enhanced tumor regression in HT29 human colon adenocarcinoma xenografts compared to each agent alone, and showed prolonged survival of animals in a disseminated model of AML in combination therapy with cytarabine. Onvansertib has favorable pharmacologic parameters and good oral bioavailability in rodent and nonrodent species, as well as proven antitumor activity in different nonclinical models using a variety of dosing regimens, which can provide a high degree of flexibility in dosing schedules, warranting investigation in clinical settings. Onvansertib has several advantages over volasertib (BI6727, another PLK1 inhibitor), including a higher degree of potency and specificity for the PLK1 isozyme, and oral bioavailability. In addition, onvansertib has proven antitumor activity in different nonclinical models using a variety of dosing regimens, which can provide flexibility in dosing schedules, and therefore, warrants investigation in clinical settings.

A phase I, first-in-human, dose-escalation study of onvansertib in patients with advanced/metastatic solid tumors identified neutropenia and thrombocytopenia as the primary dose-limiting toxicities. These hematologic toxicities were anticipated on the basis of the mechanism of action of the drug and were reversible, with recovery occurring within 3 weeks. The half-life of onvansertib was established between 20 and 30 hours. The oral bioavailability of onvansertib plus its short half-life provide the opportunity for convenient, controlled, and flexible dosing schedules with the potential to minimize toxicities and improve the therapeutic window. Pharmacodynamics and biomarker studies, including baseline genomic profiling, serial monitoring of mutant allele fractions in plasma, and the extent of PLK1 inhibition in circulating blasts, have been performed to identify biomarkers associated with clinical response and are described in PCT Application No. PCT/US2021/013287, the content of which is incorporated herein by reference in its entirety.

The major metabolic pathways found in the different animal species were N-oxidation of the N methyl-piperazine ring to give N-oxide M2 and hydroxylation on an aliphatic carbon atom of the methylene bridge of the pyrazoloquinazoline moiety to give metabolite M1. Qualitatively, no marked differences in the metabolism of onvansertib were observed between species and, quantitatively, some differences were observed cross-species.

The potential inhibitory capacity of onvansertib towards the major human cytochrome P450 (CYP) isoforms that are responsible for hepatic drug metabolism in human (CYP1A2, CYP2C8, CYP2C9, CYP2C19, CYP2D6, and CYP3A4) was investigated using human liver microsomes. Onvansertib was able to inhibit the metabolic activities of CYP2C8, CYP2C9, CYP2C19, CYP2D6, and CYP3A4 isoforms to different extents, with 50% inhibitory concentration (IC50) values ranging from 20 μM to 66 μM (Table 1). No significant inhibitory effects against CYP1A2 were detected. Considering that the concentrations relevant to achieve significant anti-tumoral activity of the compound in mice were in the order of 1 μM, the likelihood that onvansertib would show clinically relevant metabolic drug-drug interactions is considered low. In Table 1, IC₅₀ is shown as mean±SEM (standard error of mean).

TABLE 1
SUMMARY OF MEAN INHIBITOR POTENCY OF ONVANSERTIB
FOR HUMAN LIVER CYTOCHROME P450S

| P450 Enzyme | Enzyme Reaction | IC₅₀ (μM) |
|---|---|---|
| CYP1A2 | Tacrine 1-hydroxylation | >100 |
| CYP2C8 | Paclitaxel 6-hydroxylation | 20.2 ± 1.6 |
| CYP2C9 | Diclofenac 4-hydroxylation | 20.4 ± 3.2 |
| CYP2C19 | Mephenytoin 4-hydroxylation | 36.9 ± 15.7 |
| CYP2D6 | Bufuralol 1-hydroxylation | 26.8 ± 5.4 |
| CYP3A4 | Testosterone 6β-hydroxylation | 52.7 ± 9.8 |
| CYP3A4 | Midazolam 1′-hydroxylation | 66.2 ± 4.0 |

To date, a single Phase 1 safety study with onvansertib has been completed in adult patients with advanced/metastatic solid tumors at a single study site in the U.S. First cycle dose-limiting toxicities (DLTs) and the maximum tolerated dose (MTD) of onvansertib administered orally for 5 consecutive days every 3 weeks (i.e., a 21-day treatment cycle) was conducted. Safety profile of onvansertib has been determined, to determine the pharmacokinetics (PK) of onvansertib in plasma (at the MTD), and to document any antitumor activity. In one study, a total of 21 patients were enrolled, and 19 patients were treated. No DLTs occurred at the first 3 dose levels (doses of 6 mg/m²/day, 12 mg/m²/day, and 24 mg/m²/day). At the subsequent dose level (dose of 48 mg/m²/day), 2 of 3 patients developed DLTs. An intermediate dose level of 36 mg/m²/day was investigated. At the intermediate dose level, 4 patients were treated and 2 DLTs were observed. After further cohort expansion, the MTD was determined to be 24 mg/m²/day. The best observed treatment response was stable disease (SD), which occurred in 5 of the 16 evaluable patients. The study identified thrombocytopenia and neutropenia as the primary toxicities. This is consistent with the expected mechanism of action of onvansertib and with results from the preclinical studies. These hematologic toxicities were reversible, with recovery usually occurring within 3 weeks. No other clinically relevant safety findings emerged with treatment with onvansertib as a single agent. Other mechanism-related, possibly expected events such as gastrointestinal disorders, mucositis, and alopecia were not observed, confirming that with this schedule, the bone marrow is the most sensitive target of onvansertib in humans.

As disclosed herein, a combinational therapy using a MTDP inhibitor (including paclitaxel) and a PLK1 inhibitor (including onvansertib) is expected to result in significantly enhanced efficacy against cancer (e.g., prostate cancer, head and neck cancer, non-small cell lung cancer, intrahepatic cholangiocarcinoma, gastric cancer, urothelial cancer, small cell lung cancer, breast cancer, endometrial cancer, cervical cancer, rhabdomyosarcoma, cholangiocarcinoma, ovarian cancer, or a combination thereof), causing tumor regression and improved cancer survival. The resulted tumor regression and cancer survival rate/duration by the combination can be surprisingly synergistic (i.e., more than additive, superior to the cumulated anti-tumor efficacy caused by the MTDP inhibitor and the PLK1 inhibitor separately). For example, as described herein, onvansertib in combination with paclitaxel showed synergy in in vitro and in vivo models of chemo resistant ovarian carcinoma. Treatment with onvansertib and paclitaxel surprisingly synergized in triple-negative breast cancer models. Moreover, when combined together, surprisingly, the concentration of PLK1 inhibitor to obtain complete cell inhibition was significantly lower compared to that of the single agent used to obtain the same inhibition.

Provided herein include methods, compositions and kits for treating cancer in a subject (for example, a human patient suffering from cancer). The method comprises administrating a MTDP inhibitor and a PLK1 inhibitor to the patient in a manner sufficient to inhibit or reduce progression of the cancer. For example, the MTDP inhibitor and the PLK1 inhibitor can be administrated to a subject with cancer simultaneously, separately, or sequentially. It is expected that combination treatment using onvansertib and MTDP inhibitor is significantly more effective than the combination treatment using another PLK inhibitor B12536 and MTDP for various cancer treatment, including the treatment for prostate cancer and lung cancer (e.g., neuroendocrine prostate cancer).

In some embodiments, the inhibition or reduction of cancer progression is not merely additive, but is enhanced or synergistic (that is, the inhibition is greater than the combined inhibition of progression caused by the MTDP inhibitor alone plus the PLK1 inhibitor alone). The enhanced or synergistic efficacy or inhibition of any combination of a MTDP inhibitor and a PLK1 inhibitor of the present disclosure can be different in different embodiments. In some embodiments, the enhanced or synergistic efficacy or inhibition of any combination of a MTDP inhibitor and a PLK1 inhibitor of the present disclosure is, is about, is at least, is at least about, is at most, or is at most about, 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, or a number or a range between any two of these values, higher than the combined inhibition of progression caused by the MTDP inhibitor alone plus the PLK1 inhibitor alone.

The molar ratio of the PLK1 inhibitor (e.g., onvansertib) to the MTDP inhibitor (e.g., paclitaxel) can be, for example, about 1:200, 1:100, 1:90, 1:80, 1:70, 1:60, 1:50, 1:40, 1:30, 1:20, 1:10, 1:1, 10:1, 20:1, 30:1, 40:1, 50:1, 100:1, 1000:1, 2000:1, or 5000:1, or a number or a range between any two of these values. In some embodiments, the enhanced or synergistic efficacy or inhibition of cancer progression caused by a combination of the MTDP inhibitor (e.g., paclitaxel) and the PLK1 inhibitor (e.g., onvansertib) is, is about, is at least, is at least about, is at most, or is at most about 20%, 30%, 40%, 50%, 60%, 70%, 80%, 90%, 100%, 110%, 120%, 130%, 140%, 150%, 160%, 170%, 180%, 190%, 200%, 250%, 300%, or a number or a range between any two of these values, higher than the combined inhibition of progression caused by the MTDP inhibitor (e.g., paclitaxel) alone plus the PLK1 inhibitor (e.g., onvansertib) alone. For example, a combination of the MTDP inhibitor and the PLK1 inhibitor can cause a 50%, 60%, 70%, 80%, 90%, or more, inhibition of cancer progression (cancer cell viability of 50%, 40%, 30%, 20%, 10%, or less), whereas under the same conditions the combined inhibition of the MTDP inhibitor (e.g., paclitaxel) alone plus the PLK1 inhibitor alone can be 10%, 20%, 25%, 30%, or less inhibition of cancer progression (cancer cell viability of 90%, 80%, 75%, 70%, or more). Thus, the enhanced or synergistic efficacy or inhibition of cancer progression caused by the combination of the MTDP inhibitor (e.g., paclitaxel) and the PLK1 inhibitor (e.g., onvansertib) is, for example, 50%, 60%, 70%, 80%, 90%, 100%, or more higher than the combined inhibition of progression caused by the MTDP inhibitor (e.g., paclitaxel) alone plus the PLK1 inhibitor alone. In some embodiments, the MTDP inhibitor is paclitaxel and the PLK1 inhibitor is onvansertib.

The method disclosed herein can be effective with various cancer, for example, breast cancer; pancreatic cancer; gastric cancer; gastroesophageal cancer; esophageal cancer; lung cancer; prostate cancer; cervical cancer; colorectal cancer; thyroid cancer; bladder cancer; head and neck cancer; brain and central nervous system cancer; liver cancer; gallbladder cancer; cholangiocarcinoma; ovarian cancer; vaginal cancer; colorectal cancer; renal cancer; endometrial cancer; skin cancer; testicular cancer; thymus cancer; unspecified cancer; adenocarcinoma; leukemia; lymphoma; sarcoma; other neoplastic malignancies, or a combination thereof.

As described herein, the patient can achieve complete response or partial response after treatment with the MTDP inhibitor and the PLK1 inhibitor. In some embodiments, the patient achieves a complete response. In some embodiments, the patient achieves a partial response. In some embodiments, the patient did not respond to treatment with MTDP inhibitor(s) (without a PLK1 inhibitor). In some embodiments, the patient did not respond to treatment with the MTDP inhibitor alone.

The MTDP inhibitor and the PLK1 inhibitor can be administered to the patient in any manner deemed effective to treat the cancer. The MTDP inhibitor can be administered together with, or separately from, the PLK1 inhibitor. When administered separately, the MTDP inhibitor can be administered before or after the PLK1 inhibitor, or in different administration cycles.

The MTDP inhibitor and the PLK1 inhibitor can each be administered in any schedule, e.g., once or multiple times per day or week; once, twice, three times, four times, five times, six times or seven times (daily) per week; for one or multiple weeks; etc. The PLK1 inhibitor (e.g., onvansertib) can be, for example, administered orally. The MTDP inhibitor (e.g., paclitaxel) can be, for example, administered by intravenous infusion (e.g., over about 30 minutes). In some embodiments, the PLK1 inhibitor (e.g., onvansertib) is, or is only, administered to a patient daily for 14, 15, 16, 17, 18, 19, 20, 21, 22, 23, 24, 25, 26, 27, or 28 consecutive days during a cycle, for example, on the first 14, 15, 16, 17, 18, 19, 20, 21, 22, 23, 24, 25, 26, 27, or 28 consecutive days of the cycle; and the MTDP inhibitor (e.g., paclitaxel) is, or is only, administered to the patient once in each of the weeks that onvansertib is administered. The cycle can be, for example 21-28 days in length. In some embodiments, the PLK1 inhibitor is administered to a patient daily for the first 21 consecutive days during a 28-day cycle, and the patient is administered with the MTDP inhibitor once a week for the first three weeks in the 28-day cycle. In some embodiments, no PLK1 inhibitor nor MTDP inhibitor is administered to the patient in the last 7 days of the 28-day cycle. The patient can undergo one or more cycles of treatment/administration, for example at least two cycles of treatment/administration. The administration schedule of the MTDP inhibitor and the PLK1 inhibitor can be the same or different in each of the cycles of treatment/administration.

The MTDP inhibitor can be administered to the patient at any appropriate dosage, e.g., a dosage of about, at least or at most 5 mg/m², 10 mg/m², 15 mg/m², 20 mg/m², 25 mg/m², 30 mg/m², 35 mg/m², 40 mg/m², 45 mg/m², 50 mg/m², 55 mg/m², 60 mg/m², 65 mg/m², 70 mg/m², 75 mg/m², 80 mg/m², 85 mg/m², 90 mg/m², 95 mg/m², 100 mg/m², 105 mg/m², 110 mg/m², 115 mg/m², 120 mg/m², 125 mg/m², 130 mg/m², 135 mg/m², 140 mg/m², 145 mg/m², 150 mg/m², 155 mg/m², 160 mg/m², 165 mg/m², 170 mg/m², 175 mg/m², 180 mg/m², 185 mg/m², 190 mg/m², 195 mg/m², 200 mg/m², 205 mg/m², 210 mg/m², 215 mg/m², 220 mg/m², 225 mg/m², 230 mg/m², 235 mg/m², 240 mg/m², 245 mg/m², 250 mg/m², 255 mg/m², 260 mg/m², 265 mg/m², 270 mg/m², 275 mg/m², 280 mg/m², 285 mg/m², or a number between any two of these values. The dosage unit based on the body weight (mg/kg) can be converted to another unit (e.g., mg/m²) using a conversion chart such as the body surface area (BSA) conversion chart as will be understood by a person of skill in the art. In some embodiments, the MTDP inhibitor is paclitaxel, which is administered at a dosage of about, at least or at most 38 mg/m², 39 mg/m², 40 mg/m², 41 mg/m², 42 mg/m², 43 mg/m², 44 mg/m², 45 mg/m², 46 mg/m², 47 mg/m², 48 mg/m², 49 mg/m², 50 mg/m², 51 mg/m², 52 mg/m², 53 mg/m², 54 mg/m², 55 mg/m², 56 mg/m², 57 mg/m², 58 mg/m², 59 mg/m², 60 mg/m², 61 mg/m², 62 mg/m², 63 mg/m², 64 mg/m², 65 mg/m², 66 mg/m², 67 mg/m², 68 mg/m², 69 mg/m², 70 mg/m², 71 mg/m², 72 mg/m², 73 mg/m², 74 mg/m², 75 mg/m², 76 mg/m², 77 mg/m², 78 mg/m², 79 mg/m², 80 mg/m², 81 mg/m², 82 mg/m², 83 mg/m², 84 mg/m², 88 mg/m², 86 mg/m², 87 mg/m², 88 mg/m², 89 mg/m², 90 mg/m², or a number between any two of these values.

The MTDP inhibitor can be administrated to the patient once weekly or twice weekly. In some embodiments, the MTDP inhibitor is administered in a cycle of 14-28 days of daily administration. In some embodiments, the MTDP inhibitor is administered in a cycle of 14 days, 15 days, 16 days, 17 days, 18 days, 19 days, 20 days, 21 days, 22 days, 23 days, 24 days, 25 days, 26 days, 27 days, 28 days, 29 days, or 30 days. In some embodiments, the MTDP inhibitor is administered on 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days, of a cycle. In some embodiments, the MTDP inhibitor is administered on day 1, day 2, day 3, day 4, day 5, day 6, day 7, day 8, day 9, day 10, day 11, day 12, day 13, day 14, day 15, day 16, day 17, day 18, day 19, day 20, day 21, day 22, day 23, day 24, day 25, day 26, day 27, day 28, day 29, and/or day 30. In some embodiments, the MTDP inhibitor is not administered on day 1, day 2, day 3, day 4, day 5, day 6, day 7, day 8, day 9, day 10, day 11, day 12, day 13, day 14, day 15, day 16, day 17, day 18, day 19, day 20, day 21, day 22, day 23, day 24, day 25, day 26, day 27, day 28, day 29, and/or day 30. For example, paclitaxel can be administered in a cycle of 5, 6, 7, 8, 9, or 10 days. Paclitaxel can be administrated weekly in each week or in selected weeks of the administration cycle. In some embodiments, paclitaxel is administered in a cycle of 28 days with a weekly administration for three weeks (e.g., on Days 1, 8 and 15) and no administration for the remaining days of the cycle, including Days 16-28.

Similarly, any PLK1 inhibitor, now known or later discovered, can be used in these methods, including PLK1 inhibitors that are selective for PLK1, and PLK1 inhibitors that also inhibit the activity of other proteins. In some embodiments, the PLK1 inhibitor is a dihydropteridinone, a pyridopyrimidine, a aminopyrimidine, a substituted thiazolidinone, a pteridine derivative, a dihydroimidazo[1,5-f]pteridine, a metasubstituted thiazolidinone, a benzyl styryl sulfone analogue, a stilbene derivative, or a combination thereof. In some of these embodiments, the PLK1 inhibitor is onvansertib, BI2536, Volasertib (BI 6727), GSK461364, AZD1775, CYC140, HMN-176, HMIN-214, rigosertib (ON-01910), MLN0905, TKM-080301, TAK-960 or Ro3280.

In some embodiments, the PLK1 inhibitor is onvansertib. In these embodiments, onvansertib is administered to the patient at any appropriate dosage, e.g., a dosage of less than 12 mg/m², less than or equal to 24 mg/m², or greater than 24 mg/m². In some embodiments, onvansertib is administered to the patient daily. In some embodiments, onvansertib is administered in a cycle of 5-14 days of daily onvansertib administration with 2-16 days with no onvansertib administration. For example, in some embodiments, onvansertib is administered daily for 21 consecutive days followed by no onvansertib administration for 7 days in a cycle. In some embodiments, the combination treatment with onvansertib and MTDP inhibitor can be administered at the same dose as single treatment with onvansertib or MTDP inhibitor.

As can be appreciated by one of skill in the art, the amount of co-administration of the MTDP inhibitor and the PLK1 inhibitor, and the timing of co-administration, can depend on the type (species, gender, age, weight, etc.) and condition of the subject being treated and the severity of the disease or condition being treated. The MTDP inhibitor and the PLK1 inhibitor can be formulated into a single pharmaceutical composition, or two separate pharmaceutical compositions. The active ingredients can also be entrapped in microcapsules prepared, for example, by coacervation techniques or by interracial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions.

Methods, compositions, kits and systems disclosed herein can be applied to different types of subjects. For example, the subject can be a subject receiving a cancer treatment, a subject at cancer remission, a subject who has received one or more cancer treatment, or a subject suspected of having cancer. The subject can have a stage I cancer, a stage II cancer, a stage III cancer, and/or a stage IV cancer. The cancer can be head and neck cancer, non-small cell lung cancer, intrahepatic cholangiocarcinoma, gastric cancer, urothelial cancer, small cell lung cancer, endometrial cancer, cervical cancer, rhabdomyosarcoma, cholangiocarcinoma, liver cancer, ovarian cancer, breast cancer, prostate cancer, colorectal cancer, pancreatic cancer, or a combination thereof. The cancer can be an unresectable locally advanced or metastatic disease. The methods can further comprise administering an additional therapeutic intervention to the subject. The additional therapeutic intervention can comprise a different therapeutic intervention than administering the PLK1 inhibitor and the MTDP inhibitor, such as an antibody, an adoptive T cell therapy, a chimeric antigen receptor (CAR) T cell therapy, an antibody-drug conjugate, a cytokine therapy, a cancer vaccine, a checkpoint inhibitor, a radiation therapy, surgery, a chemotherapeutic agent, or any combination thereof. The therapeutic intervention can be administered at any time of the treatment, for example at a time when the subject has an early-stage cancer. The therapeutic intervention can be more effective than if the therapeutic intervention were to be administered to the subject at a later time. Without being bound to any particular theory, it is believed that the PLK1 inhibitor (e.g., onvansertib) can sensitize cells (e.g., cancer cells) to MTDP inhibitor treatment to achieve effective cancer treatment.

### Dosing and Pharmacokinetics

The treatment of the present disclosure can comprise administration of a PLK1 inhibitor (e.g., onvansertib) for a desired duration in one or more cycles of treatment, and administration of an MTDP inhibitor.

Daily or weekly administration of an MTDP inhibitor (e.g., intravenous administration) can be at, or be about, 0.01 mg, 0.05 mg, 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1 mg, 5 mg, 10 mg, 20 mg, 50 mg, 100 mg, 200 mg, 300 mg, 400 mg, 500 mg, 600 mg, 700 mg, 800 mg, 900 mg, 1000 mg, 1100 mg, 1200 mg, or a number or a range between any two of these values. The daily or weekly dose of the MTDP inhibitor can be adjusted (e.g., increased or decreased with the range) during the treatment of the subject. The daily or weekly administration of the MTDP inhibitor can be at different amounts on different days or during different weeks. For example, the treatment can comprise daily or weekly administration of the MTDP inhibitor at 0.1 mg to 20 mg during week 1, 0.25 mg to 50 mg during week 2, 0.5 mg to 100 mg during week 3, 1 mg to 200 mg during week 4, and 2 mg to 400 mg during week 5 and beyond. For example, the treatment can comprise daily or weekly administration of the MTDP inhibitor at 0.1 mg to 100 mg on day 1, 0.2 mg to 200 mg on day 2, 0.4 mg to 400 mg on day 3, and 0.4 mg to 400 mg or 0.6 mg to 600 mg on day 4 and beyond. For example, the MTDP inhibitor is paclitaxel and is administered at a daily or weekly dose of about 0.01 mg, 0.05 mg, 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1 mg, 2 mg, 3 mg, 4 mg, 5 mg, 6 mg, 7 mg, 8 mg, 9 mg, 10 mg, or a number or a range between any two of these values.

In some embodiments, the MTDP inhibitor can be administered daily or weekly at a drug/body surface area unit dose of about 15 mg/m² to about 275 mg/m². For example, the MTDP inhibitor (e.g., paclitaxel) can be administered at, or at about 5 mg/m², 10 mg/m², 15 mg/m², 20 mg/m², 25 mg/m², 30 mg/m², 35 mg/m², 40 mg/m², 45 mg/m², 50 mg/m², 55 mg/m², 60 mg/m², 65 mg/m², 70 mg/m², 75 mg/m², 80 mg/m², 85 mg/m², 90 mg/m², 95 mg/m², 100 mg/m², 105 mg/m², 110 mg/m², 115 mg/m², 120 mg/m², 125 mg/m², 130 mg/m², 135 mg/m², 140 mg/m², 145 mg/m², 150 mg/m², 155 mg/m², 160 mg/m², 165 mg/m², 170 mg/m², 175 mg/m², 180 mg/m², 185 mg/m², 190 mg/m², 195 mg/m², 200 mg/m², 205 mg/m², 210 mg/m², 215 mg/m², 220 mg/m², 225 mg/m², 230 mg/m², 235 mg/m², 240 mg/m², 245 mg/m², 250 mg/m², 255 mg/m², 260 mg/m², 265 mg/m², 270 mg/m², 275 mg/m², 280 mg/m², 285 mg/m², or a number or a range between any two of these values. In some embodiments, the MTDP inhibitor can be administered daily or weekly at a drug/body surface area unit dose of, or about, 38 mg/m², 39 mg/m², 40 mg/m², 41 mg/m², 42 mg/m², 43 mg/m², 44 mg/m², 45 mg/m², 46 mg/m², 47 mg/m², 48 mg/m², 49 mg/m², 50 mg/m², 51 mg/m², 52 mg/m², 53 mg/m², 54 mg/m², 55 mg/m², 56 mg/m², 57 mg/m², 58 mg/m², 59 mg/m², 60 mg/m², 61 mg/m², 62 mg/m², 63 mg/m², 64 mg/m², 65 mg/m², 66 mg/m², 67 mg/m², 68 mg/m², 69 mg/m², 70 mg/m², 71 mg/m², 72 mg/m², 73 mg/m², 74 mg/m², 75 mg/m², 76 mg/m², 77 mg/m², 78 mg/m², 79 mg/m², 80 mg/m², 81 mg/m², 82 mg/m², 83 mg/m², 84 mg/m², 88 mg/m², 86 mg/m², 87 mg/m², 88 mg/m², 89 mg/m², 90 mg/m² or a number or a range between any two of these values.

Each cycle of treatment/administration can have various lengths, for example, at least 14 days, 15 days, 16 days, 17 days, 18 days, 19 days, 20 days, 21 days, 22 days, 23 days, 24 days, 25 days, 26 days, 27 days, 28 days, or more. In some embodiments, the MTDP inhibitor is administered daily, semi-weekly, or weekly for three weeks in a 28-day cycle. In exemplary embodiments, the MTDP inhibitor is administered for 1 to 10 cycles, for example 1 to 9 cycles, 1 to 8 cycles, 1 to 7 cycles, 1 to 6 cycles, 1 to 5 cycles, 1 to 4 cycles, 1 to 3 cycles, 1 to 2 cycles, or 1 cycle. The administration of the MTDP inhibitor (and/or the one or more chemotherapeutic agents) can be daily or weekly and/or with break(s) between the administrations. The break can be, for example, 1 day, 2 days, 3 days, 4 days, 5 days, 6 days, 7 days, 8 days, 9 days, 10 days, 11 days, 12 days, 13 days, 14 days, or more. In some embodiments, the breaks can be 6 days and/or 13 days. In some embodiments, the daily or weekly dose of the MTDP inhibitor can be adjusted (e.g., increased or decreased with the range) during the treatment of the subject. The daily or weekly administration of the MTDP inhibitor can be at different amounts on different days or during different weeks. For example, the treatment can comprise weekly administration of the MTDP inhibitor at 80 mg/m² on day 1, 64 mg/m² on day 8, and 48 mg/m² on day 15. For example, the treatment can comprise daily or weekly administration of the MTDP inhibitor at 0.1 mg to 20 mg during week 1, 0.25 mg to 50 mg during week 2, 0.5 mg to 100 mg during week 3, 1 mg to 200 mg during week 4, and 2 mg to 400 mg during week 5 and beyond. For example, the treatment can comprise daily or weekly administration of the MTDP inhibitor at 0.1 mg to 100 mg on day 1, 0.2 mg to 200 mg on day 2, 0.4 mg to 400 mg on day 3, and 0.4 mg to 400 mg or 0.6 mg to 600 mg on day 4 and beyond. For example, the MTDP inhibitor can be administered at a daily or weekly dose of about 0.01 mg, 0.05 mg, 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1 mg, 2 mg, 3 mg, 4 mg, 5 mg, 6 mg, 7 mg, 8 mg, 9 mg, 10 mg, or a number or a range between any two of these values. In some embodiments, the daily or weekly dose of the MTDP inhibitor can be, or be about, 0.005 mg/m², 0.01 mg/m², 0.05 mg/m², 0.1 mg/m², 0.15 mg/m², 0.2 mg/m², 0.25 mg/m², 0.3 mg/m², 0.35 mg/m², 0.4 mg/m², 0.45 mg/m², 0.5 mg/m², 0.55 mg/m², 0.6 mg/m², 0.65 mg/m², 0.7 mg/m², 0.75 mg/m², 0.8 mg/m², 0.85 mg/m², 0.9 mg/m², 0.95 mg/m², 1 mg/m², 2 mg/m², 3 mg/m², 4 mg/m², 5 mg/m², 6 mg/m², 7 mg/m², 8 mg/m², 9 mg/m², 10 mg/m², or a number or a range between any two of these values. In some embodiments, a patient is administered an effective dose of a corticosteroids (e.g., dexamethasone), a diphenhydramine, and/or H2 antagonists (e.g., cimetidine or famotidine) prior to administering the MTDP inhibitor.

A maximum concentration (Cmaₓ) of the MTDP inhibitor in a blood of the subject (during the treatment or after the treatment) when the MTDP inhibitor is administered alone or in combination with the PLK1 inhibitor can be from about 1 μg/mL (picogram per mL) to about 10 μg/mL (microgram per mL). For example, the Cmaₓ of the MTDP inhibitor in a blood of the subject when the MTDP inhibitor is administered alone or in combination with the PLK1 inhibitor can be, or be about, 1 pg/mL, 5 pg/mL, 10 pg/mL, 20 pg/mL, 30 pg/mL, 40 pg/mL, 50 pg/mL, 60 pg/mL, 70 pg/mL, 80 pg/mL, 90 pg/mL, 100 pg/mL, 150 pg/mL, 200 pg/mL, 250 pg/mL, 300 pg/mL, 350 pg/mL, 400 pg/mL, 450 pg/mL, 500 pg/mL, 1000 pg/mL, 5000 pg/mL, 10000 pg/mL, 50000 pg/mL, 100000 pg/mL (0.1 pg/mL), 0.2 pg/mL, 0.3 pg/mL, 0.4 pg/mL, 0.5 pg/mL, 0.6 pg/mL, 0.7 pg/mL, 0.8 pg/mL, 0.9 pg/mL, 1 pg/mL, 1.1 pg/mL, 1.2 pg/mL, 1.3 pg/mL, 1.4 pg/mL, 1.5 pg/mL, 1.6 pg/mL, 1.7 pg/mL, 1.8 pg/mL, 1.9 pg/mL, 2 pg/mL, 2.1 pg/mL, 2.2 pg/mL, 2.3 pg/mL, 2.4 pg/mL, 2.5 pg/mL, 2.6 pg/mL, 2.7 pg/mL, 2.8 pg/mL, 2.9 pg/mL, 3 pg/mL, 3.1 pg/mL, 3.2 pg/mL, 3.3 pg/mL, 3.4 pg/mL, 3.5 pg/mL, 3.6 pg/mL, 3.7 μg/mL, 3.8 μg/mL, 3.9 μg/mL, 4 μg/mL, 4.1 μg/mL, 4.2 μg/mL, 4.3 μg/mL, 4.4 μg/mL, 4.5 μg/mL, 4.6 μg/mL, 4.7 μg/mL, 4.8 μg/mL, 4.9 μg/mL, 5 μg/mL, 5.1 μg/mL, 5.2 μg/mL, 5.3 μg/mL, 5.4 μg/mL, 5.5 μg/mL, 5.6 μg/mL, 5.7 μg/mL, 5.8 μg/mL, 5.9 μg/mL, 6 μg/mL, 6.1 μg/mL, 6.2 μg/mL, 6.3 μg/mL, 6.4 μg/mL, 6.5 μg/mL, 6.6 μg/mL, 6.7 μg/mL, 6.8 μg/mL, 6.9 μg/mL, 7 μg/mL, 7.1 μg/mL, 7.2 μg/mL, 7.3 μg/mL, 7.4 μg/mL, 7.5 μg/mL, 7.6 μg/mL, 7.7 μg/mL, 7.8 μg/mL, 7.9 μg/mL, 8 μg/mL, 8.1 μg/mL, 8.2 μg/mL, 8.3 μg/mL, 8.4 μg/mL, 8.5 μg/mL, 8.6 μg/mL, 8.7 μg/mL, 8.8 μg/mL, 8.9 μg/mL, 9 μg/mL, 9.1 μg/mL, 9.2 μg/mL, 9.3 μg/mL, 9.4 μg/mL, 9.5 μg/mL, 9.6 μg/mL, 9.7 μg/mL, 9.8 μg/mL, 9.9 μg/mL, 10 μg/mL, a range between any two of these values, or any value between 1 μg/mL to 10 μg/mL.

The treatment of the present disclosure can comprise administration of a PLK1 inhibitor (onvansertib) for a desired duration in one or more cycles. In some embodiments, the PLK1 inhibitor (e.g., onvansertib) is administered for 1 to 10 cycles, for example 1 to 9 cycles, 1 to 8 cycles, 1 to 7 cycles, 1 to 6 cycles, 1 to 5 cycles, 1 to 4 cycles, 1 to 3 cycles, 1 to 2 cycles, or 1 cycle. Each cycle of treatment can have various lengths, for example, at least 14 days, 15 days, 16 days, 17 days, 18 days, 19 days, 20 days, 21 days, 22 days, 23 days, 24 days, 25 days, 26 days, 27 days, 28 days, or more.

The administration of the PLKs inhibitor (and/or the one or more chemotherapeutic agents) can be daily or with break(s) between days of administrations. The break can be, for example, 1 day, 2 days, 3 days, 4 days, 5 days, 6 days, 7 days, 8 days, 9 days, 10 days, 11 days, 12 days, 13 days, 14 days, or more. The administration can be once, twice, three times, four times, or more on a day when the PLK1 inhibitor (and/or the one or more chemotherapeutic agents) is administered to the patient. The administration can be, for example, once every two days, every three days, every four days, every five days, every six days, or every seven days. The length of the desired duration can vary, for example, 1, 2, 3, 4, 5, 6, 7, 8, 9, 10, 11, 12, 13, 14, 15, 16, 17, 18, 19, 20, 21, 22, 23, 24, 25, 26, 27, 28, or more days. Each cycle of treatment can have various lengths, for example, at least 14 days, 15 days, 16 days, 17 days, 18 days, 19 days, 20 days, 21 days, 22 days, 23 days, 24 days, 25 days, 26 days, 27 days, 28 days, or more. For example, a single cycle of the treatment can comprise administration of the PLK1 inhibitor (e.g., onvansertib) and/or the one or more chemotherapeutic agents for four days, five days, six days, seven days, eight days, nine days, ten days, eleven days, twelve days, thirteen days, fourteen days, fifteen days, sixteen days, seventeen days, eighteen days, nineteen days, twenty days, twenty-one days, twenty-two days, twenty-three days, twenty-four days, twenty-five days, twenty-six days, twenty-seven days, twenty-eight days, or more in a cycle (e.g., in a cycle of at least 21 days (e.g., 21 to 28 days)). In some embodiments, the treatment can comprise administration of the PLK1 inhibitor (e.g., onvansertib) and/or the one or more chemotherapeutic agents for, or for at least, four days, five days, six days, seven days, eight days, nine days, ten days, eleven days, twelve days, thirteen days, fourteen days, fifteen days, sixteen days, seventeen days, eighteen days, nineteen days, twenty days, or a range between any two of these values, in a cycle (e.g., a cycle of at least 21 days (e.g., 21 to 28 days)). The administration of the PLK1 inhibitor (e.g., onvansertib) and/or the one or more chemotherapeutic agents in a single cycle of the treatment can be continuous or with one or more intervals (e.g., one day or two days of break). In some embodiments, the treatment comprises administration of the PLK1 inhibitor (e.g., onvansertib) for five days in a cycle of 21 to 28 days. In some embodiments, the PLK1 inhibitor (e.g., onvansertib) is administered daily for 21 days, followed by a 7-day break. In some embodiments, the PLK1 inhibitor (e.g., onvansertib) is administered orally. In some embodiments the PLK1 inhibitor (e.g., onvansertib) is administered without any catch-up doses.

In some embodiments, the PLK1 inhibitor (e.g., onvansertib) is administered to the subject in need thereof on twenty days (e.g., Days 1-10 and 15-24) during a 28-day cycle. The twenty days can be, for example, a continuous daily administration for ten days (e.g., Days 1-10) and another continuous daily administration (e.g., Days 15-24) for ten days, or a continuous daily administration for four sets of five days (e.g., Days 1-5, 8-12, 15-19, and 22-26). In some embodiments, the PLK1 inhibitor (e.g., onvansertib) is administered to the subject in need thereof on twenty-one days (e.g., Days 1-21) during a 28-day cycle. In some embodiments, for example when the patient is identified to have low tolerance to the PLK1 inhibitor (e.g., onvansertib), the PLK1 inhibitor is administered to the subject in need thereof on ten days (e.g., Days 1-5 and 15-19) during a 28-day cycle. The ten days can be, for example, a continuous daily administration for ten days (e.g., Days 1-10) or two continuous daily admiration for five days each (e.g., Days 1-5 and Days 15-19). In some embodiments, the PLK1 inhibitor (e.g., onvansertib) is administered to the subject in need thereof daily throughout the whole cycle (e.g., daily for 28 days in a cycle of 28 days). Depending on the needs of inhibition/reversion of cancer progression in the subject, the subject can receive one, two, three, four, five, six, or more cycles of treatment. For combination treatment, the administration cycles, dosing schedules, and/or dosage amounts of the MTDP inhibitor and the PLK1 inhibitor can be the same or different. For combination treatment, the administration cycle, dosing schedule, and/or dosage amount of the MTDP inhibitor can be adjusted according to the administration cycle, dosing schedule, and/or dosage amount of the PLK1 inhibitor. For example, the MTDP inhibitor (e.g., paclitaxel) can be administered three times in a 28-day cycles (e.g., daily dose on Days 1, 8 and 15), which corresponds to a 28-day cycle for administration of the PLK1 inhibitor (e.g., onvansertib).

The treatment can comprise administration of the PLK1 inhibitor (e.g., onvansertib) at, or at about, 6 mg/m²-90 mg/m² drug/body surface area, for example, as a daily dose. For example, the treatment can comprise daily administration of the PLK1 inhibitor (e.g., onvansertib) at, or at about, 6 mg/m², 8 mg/m², 10 mg/m², 12 mg/m², 14 mg/n², 16 mg/m², 18 mg/m², 20 mg/m², 23 mg/m², 27 mg/m², 30 mg/m², 35 mg/m², 40 mg/m², 45 mg/m², 50 mg/m², 55 mg/m², 60 mg/m², 65 mg/m², 70 mg/m², 80 mg/m², 85 mg/m², 90 mg/m², a number or a range between any two of these values, or any value between 8 mg/m²-90 mg/m². In some embodiments, the daily dose of the PLK1 inhibitor (e.g., onvansertib) can be adjusted (e.g., increased or decreased with the range) during the treatment, or during a single cycle (e.g., the first cycle, the second cycle, the third cycle, and a subsequent cycle) of the treatment, for the subject. In some embodiments, the PLK inhibitor (e.g., onvansertib) is administered at 12 mg/m² on twenty days (e.g., Days 1-10 and 15-24) during a 28-day cycle. In some embodiments, the PLK inhibitor (e.g., onvansertib) is administered at 15 mg/m² on ten days (e.g., Days 1-5 and 15-19) during a 28-day cycle. In some embodiments, the PLK inhibitor (e.g., onvansertib) is administered at 8 mg/m² or 10 mg/m² everyday (e.g., Days 11-28) during a 28-day cycle. In some embodiments, the daily dose of the PLK1 inhibitor (e.g., onvansertib) can be adjusted (e.g., increased or decreased with the range) during the treatment, or during a single cycle (e.g., the first cycle, the second cycle, the third cycle, and a subsequent cycle) of the treatment, for the subject.

A maximum concentration (Cₘₐₓ) of the PLK1 inhibitor (e.g., onvansertib) in a blood of the subject (during the treatment or after the treatment) when the PLK1 inhibitor is administered alone or in combination with the MTDP inhibitor can be from about 100 nmol/L to about 1500 nmol/L. For example, the Cₘₐₓ of the PLK1 inhibitor (e.g., onvansertib) in a blood of the subject when the PLK1 inhibitor is administered alone or in combination with the MTDP inhibitor can be, or be about, 100 nmol/L, 200 nmol/L, 300 nmol/L, 400 nmol/L, 500 nmol/L, 600 nmol/L, 700 nmol/L, 800 nmol/L, 900 nmol/L, 1000 nmol/L, 1100 nmol/L, 1200 nmol/L, 1300 nmol/L, 1400 nmol/L, 1500 nmol/L, a range between any two of these values, or any value between 200 nmol/L to 1500 nmol/L.

An area under curve (AUC) of a plot of a concentration of the PLK1 inhibitor (e.g., onvansertib) in a blood of the subject over time (e.g., AUC₀₋₂₄ for the first 24 hours after administration) when the PLK1 inhibitor is administered alone or in combination with the MTDP inhibitor can be from about 1000 nmol/L·hour to about 400000 nmol/L·hour. For example, the AUC of a plot of a concentration of the PLK1 inhibitor (e.g., onvansertib) in a blood of the subject over time (e.g., AUC₀₋₂₄ for the first 24 hours after administration) when the PLK1 inhibitor is administered alone or in combination with the MTDP inhibitor can be, or be about, 1000 nmol/L·hour, 5000 nmol/L·hour, 10000 nmol/L·hour, 15000 nmol/L·hour, 20000 nmol/L·hour, 25000 nmol/L·hour, 30000 nmol/L·hour, 35000 nmol/L·hour, 40000 nmol/L·hour, a range between any two of these values, or any value between 1000 nmol/L·hour and 400000 nmol/L·hour.

A time (Tₘₐₓ) to reach a maximum concentration of the PLK1 inhibitor (e.g., onvansertib) in a blood of the subject when the PLK1 inhibitor is administered alone or in combination with the MTDP inhibitor can be from about 1 hour to about 5 hours. For example, the time (Tₘₐₓ) to reach a maximum concentration of the PLK1 inhibitor (e.g., onvansertib) in a blood of the subject when the PLK1 inhibitor is administered alone or in combination with the MTDP inhibitor can be, or be about, 1 hour, 1.5 hours, 2 hours, 2.5 hours, 3 hours, 3.5 hours, 4 hours, 4.5 hours, 5 hours, a range between any two of these values, or any value between 1 hour and 5 hours.

An elimination half-life (T_{1/2}) of the PLK1 inhibitor (e.g., onvansertib) in a blood of the subject when the PLK1 inhibitor is administered alone or in combination with the MTDP inhibitor can be from about 10 hours to about 60 hours. For example, the elimination half-life (T_{1/2}) of the PLK1 inhibitor (e.g., onvansertib) in a blood of the subject when the PLK1 inhibitor is administered alone or in combination with the MTDP inhibitor can be, or be about, 10 hours, 15 hours, 20 hours, 25 hours, 30 hours, 35 hours, 40 hours, 45 hours, 50 hours, 55 hours, 60 hours, a range between any two of these values, or any value between 10 hours and 60 hours.

Patients administered one or more dose cycles of the MTDP inhibitor in combination with one or more dose cycles of PLK1 inhibitor can exhibit very tolerable AE, including in some cases undetectable definite AE or definite SAE. A remarkable, but unlikely result is the finding that the patient has no probable or even possible AE or SAE. In some embodiments, treated with the combined therapy of MTDP inhibitor and PLK1 inhibitor can lead to remarkable therapeutic effect. A therapeutic effect greater than the therapeutic effect predicted from in vitro or in silico is indicative of a surprising result. A therapeutic dose lower than the therapeutic dose predicted from in vitro or in silico is indicative of a surprising result. It is expected that the combination treatment can mitigate disease progression in patients. A highly positive result is the finding that the combination therapy can lead to stable disease. A remarkable, but unlikely result is the finding of a complete response or complete remission of the cancer, a progression-free survival or an overall survival rate exceeding values predicted from in vitro or in silico analysis, or is free of any measurable lesion, free of any target lesion, or free of any malignant lymph nodes.

### Additional Cancer Therapeutics or Therapy

Methods, compositions and kits disclosed herein can be used for treating cancer, for example prostate cancer. In some embodiments, a method for treating cancer comprises administrating an MTDP inhibitor and a PLK1 inhibitor (e.g., onvansertib) to a subject (e.g., a patient) in need thereof. The method can comprise administering a therapeutically effective amount of the MTDP inhibitor and a therapeutically effective amount of the PLK1 inhibitor. The treatment can comprise administration of at least one additional cancer therapeutics or cancer therapy. The treatment can comprise administration a therapeutically effective amount of at least one additional cancer therapeutics or cancer therapy. The MTDP inhibitor and the cancer therapeutics or cancer therapy can, for example, co-administered simultaneously or sequentially. The PLK1 inhibitor (e.g., onvansertib) and the cancer therapeutics or cancer therapy can, for example, co-administered simultaneously or sequentially.

### Methods for Predicting/Determining Treatment Efficacy and Status for Cancer

Also disclosed herein include methods, compositions, kits, and systems for predicting/determining clinical outcome for a combination treatment of cancer of the present disclosure, monitoring of the combination treatment, predicting/determining responsiveness of a subject to the combination treatment, determining the status of the cancer in a subject, and improving combination treatment outcome. The methods, compositions, kits and systems can be used to guide the combination treatment, provide combination treatment recommendations, reduce or avoid unnecessary ineffective combination treatment for patients. ctDNA can be analyzed to predict/determine clinical outcome for cancer treatment using a combination of an MTDP inhibitor and a PLK1 inhibitor of the present disclosure, monitor the combination treatment, predict/determine responsiveness of a subject to the combination treatment, determine cancer status in a subject, improve combination treatment outcome, guide combination treatment, provide combination treatment recommendations, and/or reduce or avoid ineffective combination treatment. ctDNA can be analyzed to predict/determine clinical outcome for cancer treatment, monitor cancer treatment, predict/determine responsiveness of a subject to a cancer treatment, determine cancer status in a subject, improve cancer treatment outcome, guide cancer treatment, provide treatment recommendations, and/or reduce or avoid ineffective cancer treatment. Such analysis of ctDNA has been described in PCT Application No. PCT/US2021/013287, the content of which is incorporated herein by reference in its entirety.

A method of determining responsiveness of a subject to a combination treatment comprising an MTDP inhibitor and a PLK1 inhibitor of the disclosure can comprise, for example, analyzing circulating tumor DNA (ctDNA) of a subject with cancer, that is undergoing a treatment and/or has received the combination treatment, thereby determining the responsiveness of the subject to the combination treatment. In some embodiments, determining the responsiveness of the subject comprises determining if the subject is a responder of the treatment, if the subject is or is going to be in CR, or if the subject is or is going to be in partial remission (PR). For example, analyzing ctDNA can comprise detecting variant allele frequency in the ctDNA in a first sample obtained from the subject at a first time point, detecting variant allele frequency in the ctDNA obtained from the subject at one or more additional time points in one or more additional samples, and determining the difference of the variant allele frequency in ctDNA between the first and at least one of the one or more additional samples, a decrease in the variant allele frequency in at least one of the additional samples relative to the first sample indicates the subject as responsive to the cancer treatment.

In some embodiments, the first time point is prior or immediately prior to the combination treatment, and at least one of the one or more additional time points are at the end of or after at least a cycle of the combination treatment. In some embodiments, the cycle of the combination treatment is the first cycle of the combination treatment. In some embodiments, the first time point is prior or immediately prior to a first cycle of the combination treatment, and the one or more additional time points are at the end of or after a second cycle of the combination treatment.

In some embodiments, the first cycle of the combination treatment is immediately prior to the second cycle of the combination treatment. In some embodiments, the method comprises continuing the combination treatment to the subject if the subject is indicated as responsive to the combination treatment. In some embodiments, the method comprises discontinuing the combination treatment to the subject and/or starting a different combination treatment to the subject if the subject is not indicated as responsive to the combination treatment.

Disclosed herein include methods of determining cancer status of a subject, comprising analyzing circulating tumor DNA (ctDNA) of a subject, thereby determining cancer status of the subject. The subject can be a subject undergoing a current combination treatment comprising an MTDP inhibitor and a PLK1 inhibitor of the present disclosure, a subject that has received a prior combination treatment of the present disclosure, and/or a subject that is in remission for the cancer. The subject in remission for cancer can be in complete remission (CR), or in partial remission (PR).

In some embodiments, analyzing the ctDNA comprises detecting variant allele frequency in the ctDNA. In some embodiments, analyzing the ctDNA comprises detecting variant allele frequency in the ctDNA obtained from the subject at a first time point in a first sample, detecting variant allele frequency in the ctDNA obtained from the subject at one or more additional time points in one or more additional samples, and determining the difference of the variant allele frequency in ctDNA between the first and at least one of the one or more additional samples, an increase in the variant allele frequency at the additional sample(s) relative to the first sample indicates that the subject is at risk of cancer relapse or is in cancer relapse.

In some embodiments, the first time point is prior or immediately prior to the combination treatment, and the one or more additional time points are at the end of or after at least a cycle of the combination treatment. In some embodiments, the cycle of the combination treatment is the first cycle of the combination treatment. In some embodiments, the first time point is prior or immediately prior to a first cycle of the combination treatment, and the one or more additional time points are at the end of or after a second cycle of the combination treatment. In some embodiments, the first cycle of the combination treatment is immediately prior to the second cycle of the combination treatment.

In some embodiments, the method comprises starting an additional treatment to the subject if the subject is indicated as in cancer relapse. The additional treatment can be the same or different from the current or prior combination treatment.

The variant allele frequency in ctDNA can be determined, for example, by total mutation count in the ctDNA in each of the first sample and one or more additional samples, or by the mean variant allele frequency in each of the first sample and one or more additional samples. In some embodiments, the variant allele frequency is mutant allelic frequency (MAF) for a driver mutation of the cancer (e.g., ovarian cancer, breast cancer, prostate cancer, colorectal cancer, pancreatic cancer, or a combination thereof). In some embodiments, the variant allele frequency is MAF for one or more driver mutations of the cancer (e.g., ovarian cancer, breast cancer, prostate cancer, colorectal cancer, pancreatic cancer, or a combination thereof). In some embodiments, Log₂(C₁/C₀)<a MAF threshold indicates a decrease in ctDNA MAF. C₀ is ctDNA MAF in the first sample and C₁ is ctDNA MAF in one of the additional samples. In some embodiments, the MAF threshold is, or is about, 0.01 to −0.10. In some embodiments, the MAF threshold is, or is about, 0.06. In some embodiments, the MAF threshold is, or is about, 0.05.

In some embodiments, the first sample comprises ctDNA from the subject before treatment, and the one of additional samples comprises ctDNA from the subject after treatment. In some embodiments, the driver mutation is a mutation in one of the below 75 genes ABL1, ANKRD26, ASXL1, ATRX, BCOR, BCORL1, BRAF, BTK, CALR, CBL, CBLB, CBLC, CCND2, CDC25C, CDKN2A, CEBPA, CSF3R, CUX1, CXCR4, DCK, DDX41, DHX15, DNMT3A, ETNK1, ETV6, EZH2, FBXW7, FLT3, GATA1, GATA2, GNAS, HRAS, IDH1, IDH2, IKZF1, JAK2, JAK3, KDM6A, KIT, KMT2A, KRAS, LUC7L2, MAP2K1, MPL, MYC, MYD88, NF1, NOTCHI, NPM1, NRAS, PDGFRA, PH1F6, PPM1D, PTEN, PTPN11, RAD21, RBBP6, RPS14, RUNX1, SETBP1, SF3B1, SH2B3, SLC29A1, SMC1A, SMC3, SRSF2, STAG2, STAT3, TET2, TP53, U2AF1, U2AF2, WT1, XPO1, and ZRSR2. In some embodiments, at least one of the one or more the driver mutations is a mutation in the 75 genes. In some embodiments, one or more the driver mutations are mutations in the 75 genes.

The driver mutation or at least one of the one or more driver mutations can be in a gene selected from the group consisting of TP53, ASXL1, DNMT3A, NRAS, SRSF2, TET2, SF3B1, FLT3, FLT3 ITD, IDH2, NPM1, RUNX1, CDKN2A, KRAS, STAG2, CALR, CBL, CSF3R, DDX41, GATA2, JAK2, PH1F6, and SETBP1. In some embodiments, the driver mutation or at least one of the one or more driver mutations is in a gene selected from the group consisting of DNMT3A, TET2, NPM1, SRSF2, NRAS, CDKN2A, SF3B1, FLT3, ASXL1, SRSF2, IDH2, NRAS, and SF3B1. In some embodiments, the method further comprises determining variant allele frequency in one or more of the ctDNA, PBMCs and BMMCs of the subject.

The ctDNA can be analyzed using, for example, polymerase chain reaction (PCR), next generation sequencing (NGS), and/or droplet digital PCR (ddPCR). The sample disclosed herein can be derived from, for example, whole blood of the subject, plasma of the subject, serum of the subject, or a combination thereof. In some embodiments, the ctDNA is from whole blood of the subject, plasma of the subject, serum of the subject, or a combination thereof.

In some embodiments, the method comprises analyzing ctDNA of the subject before the treatment. In some embodiments, the treatment comprises one or more cycles, and the ctDNA is analyzed before, during and after each cycle of the treatment. Each cycle of treatment can be at least 21 days. In some embodiments, each cycle of treatment is from about 21 days to about 28 days. In some embodiments, the subject is human.

Disclosed herein include methods of improving treatment outcome for the cancer. The method can comprise: detecting variant allele frequency in circulating tumor DNA (ctDNA) obtained from a subject at a first time point in a first sample before the subject undergoes a combination treatment comprising an MTDP inhibitor and a PLK1 inhibitor of the present disclosure; detecting variant allele frequency in ctDNA obtained from the subject at one or more additional time points in one or more additional samples after the subject undergoes the combination treatment; determining the difference of the variant allele frequency in ctDNA between the first and at least one of the one or more additional samples; and continuing the combination treatment to the subject if the subject is indicated as responsive to the combination treatment, or discontinuing the combination treatment to the subject and/or starting a different cancer treatment to the subject if the subject is not indicated as responsive to the combination treatment. A decrease in the variant allele frequency in at least one of the additional samples relative to the first sample indicates the subject as responsive to the combination treatment.

Also disclosed herein include methods of treating cancer The method can comprise: administering a combination treatment comprising an MTDP inhibitor and a PLK1 inhibitor of the present disclosure to a subject in need thereof; determining a decrease, relative to a variant allele frequency in a first sample of the subject obtained at a first time point before the subject receives the combination treatment, in a variant allele frequency in a second sample of the subject obtained at a second time point after the subject receives the combination treatment; and continuing with the combination treatment. In some embodiments, the subject is a subject newly diagnosed with cancer, for example a subject that has not received any prior cancer treatment before the combination treatment. In some embodiments, the subject has received prior cancer treatment and was in remission for the cancer, for example a subject in complete remission (CR), or in partial remission (PR) after receiving the prior combination treatment.

The first time point can be, for example, prior or immediately prior to the combination treatment. The at least one of the one or more additional time points can be, for example, at the end of or after at least a cycle of the combination treatment. In some embodiments, the cycle of the combination treatment is the first cycle of the combination treatment. In some embodiments, the first time point is prior or immediately prior to a first cycle of the combination treatment, and the one or more additional time points are at the end of or after a second cycle of the combination treatment. In some embodiments, the first cycle of the combination treatment is immediately prior to the second cycle of the combination treatment.

The variant allele frequency in ctDNA can be determined, for example, by total mutation count in the ctDNA in each of the first sample and one or more additional samples, and/or by the mean variant allele frequency in each of the first sample and one or more additional samples. In some embodiments, the variant allele frequency is mutant allelic frequency (MAF) for a driver mutation of the cancer (e.g., ovarian cancer, breast cancer, prostate cancer, colorectal cancer, pancreatic cancer, or a combination thereof). In some embodiments, the variant allele frequency is mutant allelic frequency (MAF) for one or more driver mutations of the cancer (e.g., ovarian cancer, breast cancer, prostate cancer, colorectal cancer, pancreatic cancer, or a combination thereof). In some embodiments, Log₂(C₁/C₀)<a MAF threshold indicates a decrease in ctDNA MAF. C₀ is ctDNA MAF in the first sample and C₁ is ctDNA MAF in one of the additional samples. In some embodiments, the MAF threshold is −0.05.

The driver mutation can be, for example, a mutation in one of, or at least one of the one or more the driver mutations is a mutation in one of the below 75 genes ABL1, ANKRD26, ASXL1, ATRX, BCOR, BCORL1, BRAF, BTK, CALR, CBL, CBLB, CBLC, CCND2, CDC25C, CDKN2A, CEBPA, CSF3R, CUX1, CXCR4, DCK, DDX41, DHX15, DNMT3A, ETNK1, ETV6, EZH2, FBXW7, FLT3, GATA1, GATA2, GNAS, HRAS, IDH1, IDH2, IKZF1, JAK2, JAK3, KDM6A, KIT, KMT2A, KRAS, LUC7L2, MAP2K1, MPL, MYC, MYD88, NF1, NOTCHI, NPM1, NRAS, PDGFRA, PH1F6, PPM1D, PTEN, PTPN11, RAD21, RBBP6, RPS14, RUNX1, SETBP1, SF3B1, SH2B3, SLC29A1, SMC1A, SMC3, SRSF2, STAG2, STAT3, TET2, TP53, U2AF1, U2AF2, WT1, XPO1, and ZRSR2, and/or one or more the driver mutations are mutations in the 75 genes. In some embodiments, the driver mutation or at least one of the one or more driver mutations is in a gene selected from the group consisting of TP53, ASXL1, DNMT3A, NRAS, SRSF2, TET2, SF3B1, FLT3, FLT3 ITD, IDH2, NPM1, RUNX1, CDKN2A, KRAS, STAG2, CALR, CBL, CSF3R, DDX41, GATA2, JAK2, PHF6, and SETBP1. In some embodiments, the driver mutation or at least one of the one or more driver mutations is in a gene selected from the group consisting of DNMT3A, TET2, NPM1, SRSF2, NRAS, CDKN2A, SF3B1, FLT3, ASXL1, SRSF2, IDH2, NRAS, and SF3B1.

In some embodiments, the method further comprises determining variant allele frequency in one or more of the ctDNA, PBMCs and BMMCs of the subject. The variant allele frequency in ctDNA can be detected, for example, using polymerase chain reaction (PCR) or next generation sequencing (NGS). In some embodiments, the variant allele frequency in ctDNA is detected using droplet digital PCR (ddPCR).

At least one of the first sample, the one or more additional samples, and the second sample can be derived from whole blood of the subject, plasma of the subject, serum of the subject, or a combination thereof. In some embodiments, the ctDNA is from whole blood of the subject, plasma of the subject, serum of the subject, or a combination thereof.

In some embodiments, the subject whose ctDNA is analyzed is undergoing or will be undergoing treatment for the cancer. The method can comprise analyzing ctDNA of the subject before the treatment. The treatment can comprise one or more cycles, and the ctDNA is analyzed before, during and after one or more cycles of the treatment. For example, the ctDNA can be analyzed before, during and after two or more cycle of the treatment, three or more cycle of the treatment, or each cycle of the treatment. Each cycle of treatment can be at least 21 days, for example, 21 days, 22 days, 23 days, 24 days, 25 days, 26 days, 27 days, 28 days, 29 days, 30 days, or more, or a range between any two of these values. In some embodiments, each cycle of treatment is from about 21 days to about 28 days. In some embodiments, each cycle of treatment is from 21 days to 28 days. In some embodiments, the subject is human.

### Compositions and Kits

Disclosed herein include compositions and kits for treating cancer. The kit can comprise: a PLK1 inhibitor; and a manual providing instructions for co-administrating the PLK1 inhibitor with an MTDP inhibitor to a subject for treating cancer. The kit can comprise the MTDP inhibitor. The cancer can be, for example, ovarian cancer, breast cancer, prostate cancer, colorectal cancer, pancreatic cancer, or a combination thereof.

In some embodiments, the subject has cancer (e.g., head and neck cancer, non-small cell lung cancer, small-cell lung cancer, intrahepatic cholangiocarcinoma, gastric cancer, urothelial cancer, breast cancer, endometrial cancer, cervical cancer, rhabdomyosarcoma, cholangiocarcinoma, glioblastoma, low-grade glioma, thyroid carcinoma, gallbladder cancer, ovarian cancer, prostate cancer, or a combination thereof). In some embodiments, the instructions comprise instructions for co-administrating the PLK inhibitor and the MTDP inhibitor simultaneously. In some embodiments, the instructions comprise instructions for co-administrating the PLK inhibitor and the MTDP inhibitor sequentially. In some embodiments, the instructions comprise instructions for administering of the PLK1 inhibitor orally. In some embodiments, the instructions comprise instructions for administrating the MTDP inhibitor orally.

In some embodiments, the instructions comprise instructions that the subject has received a prior MTDP inhibitor treatment. In some embodiments, the instructions comprise instructions that the subject did not respond to treatment with the MTDP inhibitor alone. In some embodiments, the instructions comprise instructions that the subject is known to be resistant to a MTDP inhibitor therapy.

In some embodiments, the instructions comprise instructions that the subject has received at least one prior treatment for the cancer. In some embodiments, the prior treatment does not comprise the use of an MTDP inhibitor, a PLK inhibitor, or both. In some embodiments, the instructions comprise instructions that the subject was in remission for the cancer. In some embodiments, the subject in remission for cancer was in complete remission (CR), or in partial remission (PR).

The instructions can comprise instructions for administering each of the MTDP inhibitor and the PLK1 inhibitor to the subject in a cycle of at least twice within a week. In some embodiments, the instructions comprise instructions for administering each of the MTDP inhibitor and the PLK1 inhibitor to the subject in a cycle of at least five times within a week. In some embodiments, the instructions comprise instructions for administering the MTDP inhibitor, the PLK1 inhibitor, or both in a cycle of at least 7 days. In some embodiments, each cycle of treatment is at least about 21 days. In some embodiments, each cycle of treatment is from about 21 days to about 28 days. In some embodiments, the instructions comprise instructions for administering the PLK1 inhibitor on at least four days in the cycle. In some embodiments, the instructions comprise instructions for not administering the PLK1 inhibitor on at least one day in the cycle. In some embodiments, the instructions comprise instructions for administrating the MTDP inhibitor daily. In some embodiments, the instructions comprise instructions for administrating the MTDP inhibitor and the PLK1 inhibitor for at least two cycles.

The MTDP inhibitor can be paclitaxel or a pharmaceutically acceptable salt thereof. In some embodiments, the PLK1 inhibitor is selective and/or specific for PLK1. In some embodiments, the PLK1 inhibitor is a dihydropteridinone, a pyridopyrimidine, a aminopyrimidine, a substituted thiazolidinone, a pteridine derivative, a dihydroimidazo[1,5-f]pteridine, a metasubstituted thiazolidinone, a benzyl styryl sulfone analogue, a stilbene derivative, or any combination thereof. In some embodiments, the PLK1 inhibitor is onvansertib, BI2536, Volasertib (BI 6727), GSK461364, AZD1775, CYC140, HMN-176, HMN-214, rigosertib (ON-01910), MLN0905, TKM-080301, TAK-960 or Ro3280. In some embodiments, the PLK1 inhibitor is onvansertib.

In some embodiments, the instructions comprise instructions for administering the PLK1 inhibitor at 8 mg/m²-90 mg/m². In some embodiments, the instructions comprise instructions for administering the MTDP inhibitor at 0.01 mg-1200 mg (e.g., daily dose of at 0.01 mg-10 mg administered orally).

The methods, compositions and kits disclosed herein can also be used to sensitize cancer cells to one or more MTDP inhibitors. The method can comprise contacting cancer cells with a composition comprising a PLK1 inhibitor (e.g., onvansertib), or a pharmaceutically acceptable salt, solvate, stereoisomer thereof, thereby sensitizing the cancer cells to the one or more MTDP inhibitors. Contacting cancer cells with the composition can occur in vitro, ex vivo, in vivo, or in any combination. In some embodiments, contacting cancer cells with the composition is in a subject's body. In some embodiments, cancer cells are contacted with the composition in a cell culture. The subject can be a mammal, for example a human. The sensitization of the cancer cells can increase the responsiveness of the cancer cells to the one or more MTDP inhibitors by, or by about, 1%, 5%, 10%, 15%, 20%, 25%, 30%, 35%, 40%, 45%, 50%, 55%, 60%, 65%, 70%, 75%, 80%, 85%, 90%, 95%, or a range between any two of these values. The sensitization of the cancer cells can increase the responsiveness of the cancer cells to the one or more MTDP inhibitors by at least, or by at least about, 1%, 5%, 10%, 15%, 20%, 25%, 30%, 35%, 40%, 45%, 50%, 55%, 60%, 65%, 70%, 75%, 80%, 85%, 90%, 95%, or a range between any two of these values. The increase of the responsiveness of the cancer cells is, in some embodiments, relative to the untreated cancer cells. The sensitization of the cancer cells can increase the responsiveness of the subject having the cancer cells to one or more MTDP inhibitors by, or by about, 1%, 5%, 10%, 15%, 20%, 25%, 30%, 35%, 40%, 45%, 50%, 55%, 60%, 65%, 70%, 75%, 80%, 85%, 90%, 95%, or a range between any two of these values. The sensitization of the cancer cells can increase the responsiveness of the subject having the cancer cells to the one or more MTDP inhibitors by at least, or by at least about, 1%, 5%, 10%, 15%, 20%, 25%, 30%, 35%, 40%, 45%, 50%, 55%, 60%, 65%, 70%, 75%, 80%, 85%, 90%, 95%, or a range between any two of these values. The increase of the responsiveness of the subject having the cancer cells is, in some embodiments, relative to the subjects untreated with the composition.

The method can comprise determining sensitization of the cancer cells to the one or more MTDP inhibitors after being contacted with the PLK1 inhibitor. The method can comprise contacting the cancer cells with the one or more MTDP inhibitors concurrently and/or after being contacted with the PLK1 inhibitor. In some embodiments, contacting the cancer cells with the one or more MTDP inhibitors occurs in the body of a subject. The subject can be a mammal, for example human. The subject can be, for example, a subject that did not respond to, or is known to be resistant to, MTDP inhibitors alone. The subject can be, for example, a subject that had prior treatment with one of the one or more MTDP inhibitors. In some embodiments, the method comprises determining the response of the subject to the one or more MTDP inhibitors.

### EXAMPLES

Some aspects of the embodiments discussed above are disclosed in further detail in the following examples, which are not in any way intended to limit the scope of the present disclosure.

### Example 1

### Onvansertib Synergizes with Paclitaxel in SCLC Cell Lines

Five small cell lung cancer (SCLC) cell lines were treated with various doses of onvansertib and paclitaxel for 6 days. Cell viability was measured using CellTiterGlo® assays. Synergy of the drug combination was calculated using SynergyFinder application. The Bliss independence synergy scores for each cell line were tested in SHP77, DMS53, DMS114, H1417, and H69 cell lines, respectively. Positive Bliss independence synergy scores indicated synergy between onvansertib and paclitaxel at given concentrations. In these cell lines, at ovansertib concentration of about 25 nM, strong synergistic efficacy (as indicated by high positive synergy scores) was observed over a wide range of paclitaxel concentrations (about 0.03-500 nM). Furthermore, viability in cells treated with various doses of paclitaxel in the absence or presence of onvansertib were also tested in SHP77, DMS53, DMS114, H1417, and H69 cell lines, respectively. Expected viability was calculated using the Bliss independence model of drug additivity. The difference between the expected viability and the observed viability in the presence of onvansertib (about 25 nM) indicated synergy between onvansertib and paclitaxel. For example, in DMS53 cells, the expected and observed viabilities for combinations of onvansertib and paclitaxel are about 75% and about 50%, respectively (i.e. 25% expected, while 50% observed cancer cell inhibition), which represented an about 100% increase in efficacy with the drug combination compared to the combined inhibition caused by paclitaxel alone plus onvansertib alone. Collectively, these results demonstrated an enhanced or synergistic efficacy in inhibiting cancer progression using a combination of paclitaxel and onvansertib.

### Example 2

### Onvansertib Synergizes with Paclitaxel in TNCB Cell Lines

Activity of the PLK1 inhibitor GSK461364 alone and with the MTDP inhibitor docetaxel in several TNCB cell models was assessed. Surprisingly, the anti-tumor activity of the PLK1 inhibitor GSK461364 was significantly synergistic when combined with the MTDP inhibitor docetaxel at concentrations significantly lower than those required for single agent activity. Also, it was surprising to find that the PLK1 inhibitor GSK461364 plus the MTDP inhibitor docetaxel was able to significantly reduce the clonogenic potential and the stem cell fraction of SUM149 and SUM159 cells, two well-studied TNBC cell lines. Without being bound to a particular theory, it is believed that the fact that PLK1 is required for mitotic entry during recovery from G2 arrest induced by DNA damage. Preliminary data showed that PLK1 is a functional key gene in the basal-like cell line SUM149. The synergic combination of PLK1 inhibition and chemotherapies using, for example, a taxane, can specifically inhibit the G2-M transition, induce aberrant mitotic exit and apoptosis, and eliminate stem cell-like resistant tumor clones.

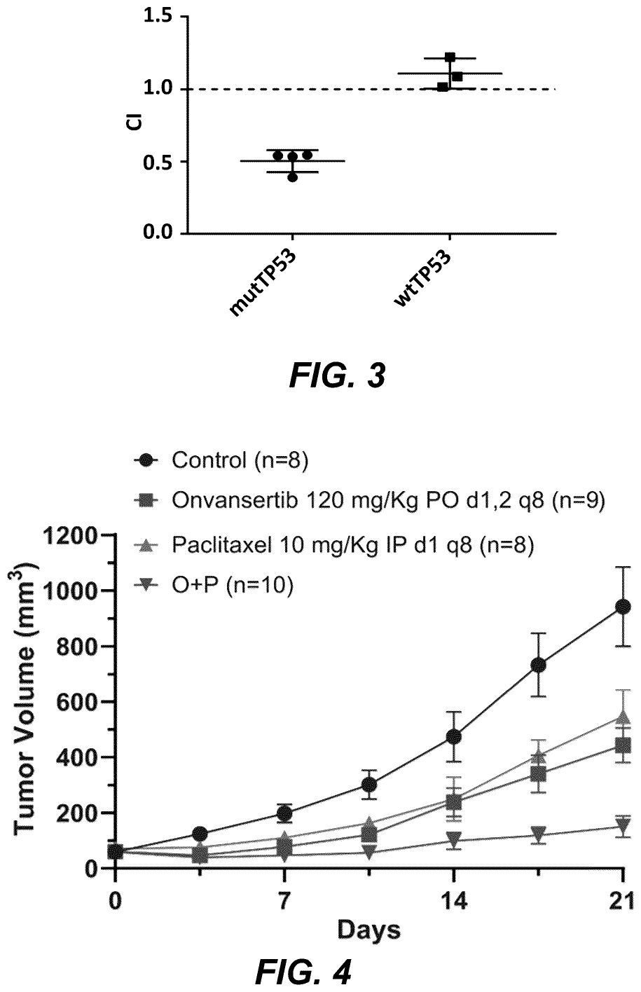

As shown in FIG. 3, the Combination Index (CI) was calculated for the combination of paclitaxel and onvansertib in cell lines with p53 mutation (TNBC: SUM149 and SUM159; Luminal: SUM52 and T47D) and wild type p53 (TNBC: SUM1315; Luminal: MCF7; Normal: MCF10A). Synergy observed (CI<1) was not dependent on TNBC vs ER+/luminal classification but rather p53 mutational status. These data suggest that mutant p53 breast cancer patients might be more sensitive to the PLK1 inhibitor onvansertib.

The effect of PLK1 inhibition via treatment with onvansertib, alone or in combination with paclitaxel, on mouse xenograft models of the mesenchymal breast cancer SUM159 was examined. A total of 35 mice were randomized to receive oral (P.O.) vehicle plus intraperitoneal (IP.) vehicle (N=8), P.O. onvansertib 120 mg/Kg on day 1 and 2 every week plus I.P. vehicle (N=9), I.P. paclitaxel 10 mg/Kg on day 1 every week plus P.O. vehicle (N=8), and P.O. onvansertib 120 mg/Kg on day 1 and 2 every week plus I.P. paclitaxel 10 mg/Kg on day 1 every week (N=10) (FIG. 4). Onvansertib and paclitaxel demonstrated similar tumor growth inhibition when compared to controls (difference=−0.406 and −0.337 with p=0.262 and 0.340, respectively, at 21 days). The combination of onvansertib and paclitaxel was significantly superior to single agent treatment with difference of −1.346 compared to onvansertib alone and −1.414 compared to paclitaxel alone (p<0.0001 and p<0.0001, respectively).

The dose and schedule of 9 mg/m² onvansertib on Day 1 to Day 21 of each 28-day treatment cycle was proposed to increase the exposure time to onvansertib and optimize efficacy. Based on PK data from the completed onvansertib Phase 1 single agent study, onvansertib concentration dropped below its effective level threshold (10×the IC₅₀ to account for effects of plasma protein binding) by Day 7 (48 hours after the last dose of onvansertib), indicating that the tumor is not exposed to onvansertib for a period of 14 days in a 21-day cycle. Extended treatment breaks can enable the tumor to recover and continue to grow.

A more continuous regimen of onvansertib was under clinical investigation in patients with mCRPC (NCT03414034). In this study, patients were treated with onvansertib 12 mg/m² QD for 14 days (Day 1 to Day 14) in combination with abiraterone QD in a 21-day cycle (beginning on Day 1 and continuing uninterrupted throughout each cycle) (14+7 regimen). As of 4 Dec. 2020, 9 patients have been treated with this regimen, and no Grade 2 or higher hematological toxicities have been reported. While the completed onvansertib Phase 1 single agent study showed that neutropenia and thrombocytopenia were the primary on-target toxicities of onvansertib, patients with mCRPC treated with onvansertib 12 mg/m² in a 14+7 regimen did not experience decreases in neutrophils and platelets. Together, these data indicated that prolonged exposure to onvansertib treatment at 12 mg/m² was safe and well tolerated. Of the 9 patients treated with onvansertib in the 14+7 regimen, 5 were evaluable for efficacy and 3 (60%) met the primary efficacy endpoint. In the alternative dosing regimen tested in the mCRPC study (5 days of onvansertib treatment at 18 mg/m² in a 14-day cycle, [5+9 regimen]), 3 (30%) of the 10 evaluable patients met the primary efficacy endpoint. Although preliminary, these data suggested that a regimen of more continuous dosing with onvansertib can increase efficacy.

Pharmacokinetic data from the completed onvansertib Phase 1 single agent study was used to simulate onvansertib drug exposure in different dosing regimens. Onvansertib effective level threshold was calculated as 10×the IC₅₀ to account for effects of plasma protein binding. For evaluating drug exposure, maximum concentration (Cₘₐₓ), area under the curve from 0 to 336 hours (AUC₍₀₋₃₃₆₎), average concentration (C_{avg}; calculated as AUC₍₀₋₃₃₆₎/336 hours) and time above 10×IC₅₀ were calculated (Table 2). In Table 2, dosing schedule represents number of days receiving onvansertib QD together with number of days not receiving onvansertib QD in any given treatment cycle. For example, a 5+9 dosing schedule represents onvansertib dosing QD on Day 1 to Day 5 of a 14-day treatment schedule. 10×IC₅₀ of onvansertib is calculated as 32.5 mg/mL to account for effects of plasma protein binding. Time above the 10×IC₅₀ was calculated for a 14-day cycle. The simulations showed that while the C_{avg} of onvansertib was increased in the more continuous dosing regimens (12 mg/m² 10+4 regimen (10 days of onvansertib treatment at 12 mg/m² in a 14-day cycle) or 14+7 regimen), the Cₘₐₓ was lower than for the 15 mg/m² 5+9 regimen. Additionally, the time above 10×IC₅₀ during a 14-day period was increased in patients treated at 12 mg/m² for 10 days compared to patients treated at 15 mg/m² for 5 days (263 hours versus 148 hours, respectively). Therefore the 12 mg/m² 10+4 regimen can result in a more profound and durable response in patients.

### Table 2: Simulated Onvansertib Pharmacokinetic Results with

DIFFERENT DOSE AND DOSING REGIMENS

| | | | | | Time |
|---|---|---|---|---|---|
| | | | | | above |
| | | | | | 10 × |
| Onvansertib | Dosing | Cₘₐₓ | C_{avg} | AUC₍₀₋₃₃₆₎ | IC₅₀ |
| dose | schedule | (ng/mL) | (ng/mL) | (ng × hr/mL) | (hr) |
| 15 mg/m² | 5 + 9 | 223 | 61.9 | 20800 | 148 |
| 12 mg/m² | 10 + 4 | 179 | 99.1 | 33300 | 263 |
| 12 mg/m² | 14 + 7 | 179 | 133 | 44600 | NA |

The dose and schedule of onvansertib for this study was selected based on analysis of pharmacokinetic (PK), pharmacodynamic, safety, and efficacy data from a Phase 1 dose escalation study. A change in the dose and schedule of onvansertib, as well as combination with paclitaxel, can alter the toxicity of the regimen. Therefore, the starting dose selected for this study was 9 mg/m²/day for 21 consecutive days, followed by 7 days off (4-week cycle). The 9 mg/m²/day starting dose allowed continuous exposure to onvansertib during weekly paclitaxel infusions.

### Example 3

### Correlative Studies Background

This example describes an investigation of PLK1 inhibition with onvansertib in combination with taxane for the treatment of TNBC patients and TP53 mutations as biomarker of response. From TCGA bioportal, TP53 is one of the most mutated gene in breast cancer. The frequency of mutations varies with the histological and biochemical characteristics of the breast cancers, being more common in ductal than lobular, in lymph-node positive than in lymph-node negative, in estrogen receptor (ER)-negative than in ER-positive, and in HER2-positive than in HER2-negative cases. As mentioned above, the prevalence of mutations also depends on the molecular subtype of the cancer, being most frequent in triple negative breast cancer (TNBC) and least frequent in the luminal A subtype. Moreover, there are several evidence that TP53 mutations were associated with a shorter OS in patients with luminal B and TNBC. At the moment, a targeted therapeutic approach for the treatment of TP53 mutated breast cancer patients does not exist, and patients receive standard chemotherapy. Since TP53 mutated breast cancers are genomically unstable and have difficulties in progressing through mitosis, PLK1 is particularly important in these cells, and hence a potential drug target. As a result, blocking PLK1 function can affect the ability of cancer cells with mutp53 to progress through mitosis, and thus can increase the overall sensitivity of the cells to chemotherapy agents such as paclitaxel. Several other genes have been involved in genomic instability of basal-like breast cancer, including RB, CCND1, BIRC5, BUB1, and PTEN. With the recent advancement of immune-oncology, PLK1 has been explored as immunomodulator in cancer treatment. Cancers with higher PLK1 expression levels tend to have lower immune activities, such as lower HLA expression and decreased B cells, NK cells and tumor-infiltrating lymphocytes infiltration. On the other hand, elevated tumor immunity increases the sensitivity of cancer cells to PLK1 inhibitors. The main mechanism underlying the associations between PLK1 and tumor immunity may lie in the aberrant cell cycle and p53 pathways in cancers. Thus, PLK1 inhibition and immunotherapy combination can achieve a synergistic antitumor efficacy.

During both phases of the study, a baseline biopsy was required if disease was safely accessible for biopsy. Samples were subjected to whole exome sequencing (WES) to capture (1) TP53 mutations as well as the mutation status of most genes involved with genomic instability (2) cell cycle proteins (Rb, CCND1, BIRC5, BUB1), and (3) overall mutational signature.

Fresh baseline biopsy samples also underwent RNA sequencing to assess gene expression and breast molecular subtypes. In the event that a subject did not undergo a baseline biopsy, or the tissue collected at this biopsy was deemed inadequate, TP53 status was performed on archival tissue.

Blood was collected at baseline, C3D1, and EOT, for circulating tumor DNA (ctDNA) assessment. In addition to evaluating genomic alterations in DNA extracted from blood, the findings were compared to the parallel sequencing analysis done on tissue biopsies from the same subjects to compare similarities and any potential differences. This analysis was to determine whether ctDNA sequencing can be a possible substitute for tissue-based sequencing which would be critical, as tissue biopsies are both invasive and costly. Quantitative changes in ctDNA was evaluated through the course of therapy to evaluate whether there were genomic alterations that can be associated with improved clinical outcome with onvansertib and paclitaxel and to assess biomarker of response. Peripheral blood mononuclear cell (PBMC) was collected and analyzed on C1D1, C1D15, and EOT for assessment of the role of onvansertib on improvement of antitumor immunity.

### Example 4

### Clinical Trial of Onvansertib and Paclitaxel Combination Treatment in Patients with TNBC with Unresectable Locally Advanced or Metastatic Disease

### Patient Selection

Patients ≥18 years old, with histologically confirmed invasive breast cancer with unresectable locally advanced or metastatic disease, having an Eastern Cooperative Oncology Group (ECOG) performance status of 0 or 1, and who was not receiving any other cancer therapy, were enrolled irrespective ethnicity, race, and gender. The invasive breast cancer with unresectable locally advanced or metastatic disease can include inflammatory breast cancer and TNBC. A histologically or cytologically-confirmed TNBC is defined as estrogen receptor ≤10%, progesterone receptor ≤10%, Her-2-neu negative per ASCO/CAP 2018 guidelines (0-1+ by IHC or FISH-negative). Patients with treated brain metastases that were stable on imaging for at least four weeks prior to registration and who were off steroid therapy were eligible. Patients with small, asymptomatic incidental brain metastases that required no immediate treatment, including steroids, were also eligible. However, concurrent endocrine therapy was not permitted for patients with estrogen receptor/progesterone receptor ≥1%. In addition, patients' organ function must be as follows.

TABLE 3
PATIENTS' ORGAN FUNCTION

| Function | Values |
|---|---|
| leukocytes | ≥3,000/mcL |
| absolute neutrophil count | ≥1,500/mcL |
| platelets | ≥100,000/mcL |
| hemoglobin | ≥9.0 g/dL |
| total bilirubin | ≤1.5 × institutional upper |
| | limit of normal (ULN) |
| AST(SGOT)/ALT(SGPT) | ≤3 × institutional ULN |
| creatinine | ≤1.5 ULN or creatinine clearance >50 |
| | ml/minute as calculated by the |
| | Cockcroft-Gault equation |

A patient was excluded if the patient: (1) received an anti-cancer chemotherapy or biologic therapy within 21 days or 5 half-lives (whichever was shorter) prior to the first dose of study drug; (2) received a palliative radiation therapy ≤2 weeks from enrollment; (3) had >3 lines of chemotherapy for metastatic disease in the phase 2 portion (no limit on prior lines in the dose escalation cohort); (4) had a disease that was relapsed or progressed less than 6 months after most recent exposure to any taxane-based therapy in neoadjuvant, adjuvant, or metastatic setting; (5) had a major surgery within 6 weeks prior to treatment initiation; (6) was pregnant or breastfeeding; (7) had a gastrointestinal (GI) disorder(s) that, in the opinion of the Investigator, can significantly impede the absorption of an oral agent (e.g., intestinal occlusion, active Crohn's disease, ulcerative colitis, extensive gastric and small intestine resection); (8) was unable or unwilling to swallow study drug; (9) had uncontrolled intercurrent illness including, but not limited to, ongoing or active infection, clinically significant non-healing or healing wounds, symptomatic congestive heart failure (CHF) Class II or higher according to the New York Heart Association (NYHA) Functional Classification, unstable angina pectoris, clinically significant cardiac arrhythmia, significant pulmonary disease (shortness of breath at rest or mild exertion), uncontrolled infection or psychiatric illness/social situations that would limit compliance with study requirements; (10) had known active infection with COVID-19 or Human Immunodeficiency Virus (HIV), with measurable viral titer, and/or active infection with hepatitis B or C (patients who had a hepatitis B virus (HBV) immunization were eligible; subjects with HIV and CD4+ T-cell (CD4+) counts ≥350 cells/μL were eligible; subjects on established ART for at least four weeks and had an HIV viral load less than 400 copies/mL prior to enrollment were eligible); (11) had clinically significant ascites or pleural effusions; (12) was known to be hypersensitivity to paclitaxel; (13) had a grade 2 or higher peripheral neuropathy; (14) had a history of other malignancies except: adequately treated non-melanoma skin cancer, curatively treated in situ cancer of the cervix, or other solid tumors curatively treated with no evidence of disease for ≥2 years; (15) had an active disease condition that would render the protocol treatment dangerous or impair the ability of the patient to receive study drug; (16) had a QT interval with Fridericia's correction (QTcF) ≥480 milliseconds. The QTcF is calculated as the arithmetic mean of the QTcF on triplicate ECGs. In the case of potentially correctible causes of QT prolongation (e.g., medications, hypokalemia), the triplicate ECG can be repeated once during screening. Result can be used to determine eligibility; (17) had a planned concomitant use of medications known to prolong the QT/QTc interval; (18) presented risk factors for torsade de pointes, including family history of Long QT Syndrome or uncorrected hypokalemia; or (19) used or planned to use strong inhibitors of CYP3A4, such as atazanavir, ceritinib, clarithromycin, cobicistat and cobicistat-containing coformulations, darunavir, idelalisib, indinavir, itraconazole, ketoconazole, lonafarnib, lopinavir, mifepristone, nefazodone, nelfinavir, ombitasvir-paritaprevir-ritonavir, ombitasvir-paritaprevir-ritonavir plus dasabuvir, posaconazole, ritonavir and ritonavir-containing coformulations, saquinavir, telithromycin, tucatinib, and voriconazole; and/or strong inducers of CYP3A4, such as apalutamide, carbamazepine, enzalutamide, fosphenytoin, lumacaftor, lumacaftor-ivacaftor, mitotane, phenobarbital, phenytoin, primidone, and rifampin (rifampicin).

### Pre-Treatment Criteria

Patients with laboratory tests meeting the following criteria are treated: (1) absolute neutrophil count >1000/mm³; (2) platelets >100,000/mm³; (3) total bilirubin of 1.5×ULN (institutional); and (4) serum creatinine <1.5×ULN (institutional) or calculated growth factor receptor (GFR) 60 mL/min.

### Onvansertib and Paclitaxel Combination Treatment

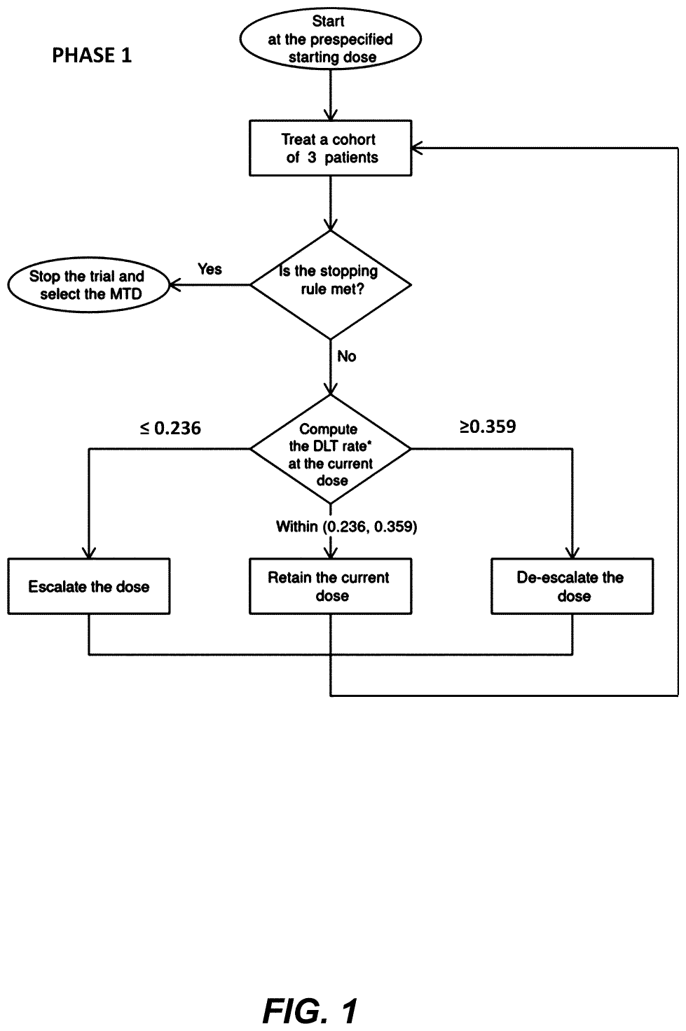

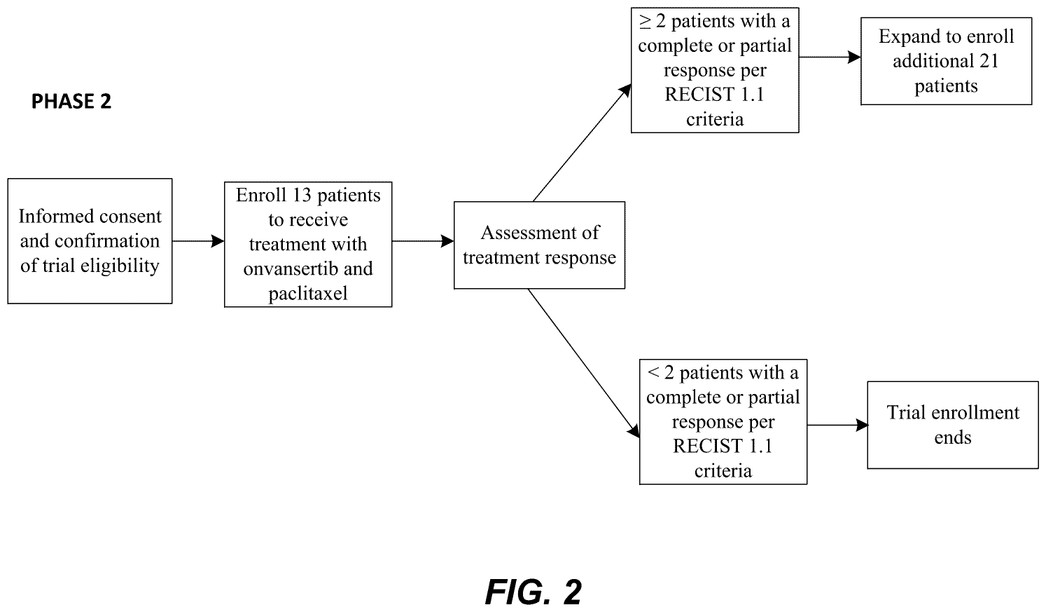

Patients meeting the pre-treatment criteria below within 72 hours of day 1 of cycle 1 were administered the onvansertib paclitaxel combination treatment in a Phase 1b clinical trial that was managed using BOIN design and a Phase 2 clinical trial that was managed using a Simon two-stage design using the recommended phase 2 dose (RP2D) for administering onvansertib. Treatment and study visits were administered within a +/−3 day window. Patients were divided into 3 cohorts with up to 9 patients treated at each dose for a cycle of 28 consecutive days, in accordance with the dose escalation/de-escalation decision map of FIG. 1, the dose escalation/de-escalation plan of Table 4, the regimen description of Table 5, and the escalation/de-escalation rules of Table 6. Starting at dose level 0, patients were administered an oral dose of onvansertib daily for the first 21 days during the 28-day cycle. Patients were also intravenously administered paclitaxel on days 1, 8, and 15 during the 28-day cycle at a dose of 80 mg/m², which can be reduced at the discretion of the principal investigator. Acceptable forms of paclitaxel included nab-paclitaxel (albumin bound paclitaxel). In Table 4, oral onvansertib administration once daily for 21 consecutive days was followed by 7 days off, to comprise a complete cycle of 28 days. In Table 6, “eliminate” means to eliminate the current and higher doses and prevent treating any future patients at these doses because the doses were overly toxic. When a dose was eliminated, doses were automatically de-escalated to the next lower level. When the lowest dose was eliminated, the trial was stopped for safety and no dose was selected as the MTD. If the current dose was the lowest dose and the rule indicated dose de-escalation, then the next patient cohort was treated at the lowest dose unless the number of DLTs reached the elimination boundary, at which point the trial was terminated for safety. If the current dose was the highest dose and the rule indicated dose escalation, the next patient cohort was treated at the highest dose. If none of the actions (i.e., escalation, de-escalation or elimination) was triggered, the new patients were treated at the current dose.

TABLE 4
DOSE ESCALATION PLAN

| Dose Level | Onvansertib Dose (QD) | Paclitaxel Dose |
|---|---|---|
| −1 | 9 mg/m² | 80 mg/m² |
| 0 (Starting dose level) | 12 mg/m² | 80 mg/m² |
| 1 | 18 mg/m² | 80 mg/m² |

TABLE 5
REGIMEN DESCRIPTION

| | | | | | Cycle |
|---|---|---|---|---|---|
| Agent | Premedication; Precautions | Dose | Route | Schedule | Length |
| Paclitaxel | Dexamethasone 20 mg PO administered | 80 mg/m² | IV | Once on | 28 days |
| | approximately 12 and 6 hours before (or 12 | | | days 1, 8, | |
| | mg IV 30 to 60 minutes before), | | | and 15 of | |
| | diphenhydramine 50 mg IV 30 to 60 minutes | | | every cycle | |
| | prior, and cimetidine (300 mg) or famotidine | | | | |
| | (20 mg) IV 30 to 60 minutes before | | | | |
| Onvansertib | Should be administered after a ≥30 minutes | As per | Oral | Daily for 21 | |
| | fast with a large glass of plain water (8 | assigned | | days, of | |
| | ounces) without ice | dose level | | every cycle | |

TABLE 6
ESCALATION/DE-ESCALATION RULES FOR THE
BOIN DESIGN WHEN TARGET DLT RATE IS 0.3

| | The number of patients treated | | | | | | | | |
|---|---|---|---|---|---|---|---|---|---|
| | at the current dose | | | | | | | | |
| | 1 | 2 | 3 | 4 | 5 | 6 | 7 | 8 | 9 |
| Escalate if # of DLT<= | 0 | 0 | 0 | 0 | 1 | 1 | 1 | 1 | 2 |
| Deescalate if # of | 1 | 1 | 2 | 2 | 2 | 3 | 3 | 3 | 4 |
| DLT>= | | | | | | | | | |
| Eliminate if # of DLT>= | NA | NA | 3 | 3 | 4 | 4 | 5 | 5 | 5 |

Onvansertib was orally administered to a patient in whole with approximately 8 ounces (approximately 240 mL) of water without ice after a more than 30 minutes of fast once every 24 hours for 21-day in a 28-day cycle in accordance with Table 5 (once daily for 21 days followed by 7 days off). Missed doses can be administered up to a maximum of 4 hours after the scheduled time. Food consumption can resume after four hours and “make up” doses should not be administered, even after vomiting. During Phase 1b, a dosing of onvansertib should be administered in clinic before dosing of paclitaxel on C1D1 (cycle 1 day 1), C1D8, C1D15, and C2D1. On all other days (including all of Phase 2), onvansertib should be administered at home or in clinic at approximately the same time each day.

All patients were pretreated with corticosteroids, diphenhydramine, and H2 antagonists. Prior to administering paclitaxel, patients were premedicated with dexamethasone, diphenhydramine, and cimetidine or famotidine as described in Table 5. Premedication for paclitaxel can start after the administration of onvansertib. Paclitaxel was administered intravenously in accordance with institutional package insert once weekly for 3 weeks of every 4 weeks. An in-line filter with a microporous membrane not greater than 0.22 microns was used.

Body surface area (BSA) for dose regimen was calculated by DuBois formula, as per DFCI policy. For paclitaxel and onvansertib, BSA was determined on day 1 of each cycle. Dose rounding for onvansertib was rounded to the nearest 5 mg. No investigational or commercial agents or therapies other than those described below can be administered with the intent to treat the participant's malignancy.

### Phase 1b Trial

The trial started at 12 mg/m² of onvansertib. If the observed dose-limiting toxicity (DLT) rate was <0.236, the next cohort of patients were treated at the next higher dose level. If the observed DLT rate was ≥0.359, the next cohort of patients were treated at the next lower dose level. The target DLT rate was 0.3 or 30%. For the purpose of overdose control, doses j and higher levels were eliminated from further examination if Pr(pj>0.3|data) ≥0.95, where pj=true DLT rate of dose level j, and j=1, . . . , 3. The trial was stopped for safety if the lowest dose was eliminated. The trial was completed when the maximum sample size of 24 was reached or 9 patients continued to receive the same dose according to the escalation/de-escalation rules of Table 6.

A DLT having a 95% confidence interval was determined in accordance with CTACE version 5.0 following the first cycle of treatment. An onvansertib maximum tolerated dose (MTL) of from 9 mg/m² to 18 mg/m² was indicative that the combination of onvansertib and paclitaxel was safe and well tolerated, and the dose can be the recommended phase 2 dose. Serious adverse effects, including grade 3 and grade 4 toxicities, were separately recorded. A proportion of patients experiencing serious adverse effects, such as grade 3 and grade 4 toxicities not exceeding 16% was indicative that the combination of onvansertib and paclitaxel was safe and well tolerated.

The RP2D is determined by isotonic regression previously specified and using the shiny app “BOIN.” The isotonic estimate of the toxicity rate closest to the target toxicity rate was selected as the RP2D. If there were ties, the higher dose level was selected when the isotonic estimate was lower than the target toxicity rate and the lower dose level was selected when the isotonic estimate was greater than or equal to the target toxicity rate.

Pharmacokinentics of the onvansertib and paclitaxel combination treatment, including the Cₘₐₓ, AUC, plasma half-life and plasma clearance, were also determined.

Patient sample size was determined by simulating 10000 trials (using shiny app “BOIN” available at www.trialdesign.org) under different scenarios. Table 7 shows that approximately 14-16 subjects was needed. Trial operating characteristics in Table 7 shows that the Phase 1b trial selected the true MTD, if any, with high probability and allocated more patients to the dose levels with the DLT rate closest to the target of 0.3.

TABLE 7
EXPECTED SAMPLE SIZE FOR DIFFERENT TOXICITY
PROFILES and TRIAL OPERATING CHARACTERISTICS

| | Dose of onvansertib | | | | |
|---|---|---|---|---|---|
| | −1 | 0 | +1 | | Expected |
| | (9 | (12 | (18 | % early | sample |
| | mg/m²) | mg/m²) | mg/m²) | stopping | size |
| Scenario 1 | | | | 0.02 | 14.8 |
| True DLT rate | 0.1 | 0.18 | 0.25 | | |
| Selection % | 2.95 | 21.63 | 75.4 | | |
| % Pts Treated | 5.7 | 42.8 | 51.5 | | |
| Scenario 2 | | | | 0.11 | 16.4 |
| True DLT rate | 0.11 | 0.3 | 0.45 | | |
| Selection % | 15.97 | 60.86 | 23.06 | | |
| % Pts Treated | 17.8 | 54.7 | 27.6 | | |
| Scenario 3 | | | | 9.8 | 15.2 |
| True DLT rate | 0.3 | 0.47 | 0.64 | | |
| Selection % | 58.61 | 30.28 | 1.31 | | |
| % Pts Treated | 43.8 | 48.1 | 8.1 | | |
| Scenario 4 | | | | 23.34 | 14.2 |
| True DLT rate | 0.4 | 0.47 | 0.64 | | |
| Selection % | 48.71 | 26.71 | 1.24 | | |
| % Pts Treated | 44.2 | 47.2 | 8.6 | | |

### Phase 2 Trial

The Phase 2 trial was a single arm, Simon's two-stage design that utilized the RECIST 1.1 criteria to determine the objective response rate of the onvansertib-paclitaxel combination treatment. The phase 2 study enrolled 1st-3rd line patients. Setting the objective response rate of paclitaxel alone in the 3rd line setting to 10%, an objective response rate of 10% was of no clinical interest and was expected for paclitaxel alone. An objective response rate of 15% was of clinical interest and the target objective response rate was 25%. A Simon's two stage design with a one-sided type I error of 10% and a power of 80% was used to detect a difference between a null of 10% ORR and a target of 25% objective response rate.

The first stage of the design started with 13 patients. A result of 2 or more response was indicative of non-futility and accrual continued to the second stage, with enrollment of 21 additional patients. A response of 6 or more among the 34 patients was indicative that the combination therapy had clinical benefit worthy of further study (exact alpha=0.095). A true response rate of 10% or 25% was indicative that there was a 62% or 13% probability that the study would discontinue after the first stage respectively.

Safety and tolerability of the onvansertib and paclitaxel combination were also evaluated. Treatment-related toxicities were summarized by maximum grade and by term using CTCAE v4.0 and were reported with 90% binomial exact confidence intervals.

The progression free survival (PFS), defined as the time from randomization (or registration) to the earlier of progression (per RECIST 1.1) or death due to any cause, was also evaluated using the RECIST 1.1 criteria for the combination treatment. Patients alive without disease progression were censored on date of last disease evaluation. PFS and overall survival (OS) were described using the methods of Kaplan-Meier and were presented with 95% confidence intervals.

Determination ofDose Limiting Toxicity (DLT)

DLTs were measured from Phase 1b following the first cycle of 28-day therapy. DLTs were defined as toxicities possibly related to the clinical trial and fitted one or more of the following criteria: (1) death not clearly due to the underlying disease or extraneous causes; (2) ≥Grade 3 non-hematologic toxicity with the following exception; (3) a change in liver function meeting Hy's Law; (4) ≥Grade 3 febrile neutropenia; (5) ≥Grade 4 neutropenia or thrombocytopenia lasting ≥7 days; and (6) ≥Grade 3 thrombocytopenia with bleeding. The exceptions of Grade 3 non-hematologic toxicity included: (1) ≥Grade 3 electrolyte abnormality with clinical symptoms, regardless of duration; (2) ≥Grade 3 nausea or vomiting or diarrhea <72 hours with adequate antiemetic or other supportive care; (3) ≥Grade 3 fatigue lasting <7 days; or (4) ≥Grade 3 electrolyte abnormality that lasts <72 hours with no clinical symptoms.

A DLT can lead to a hold of drug until the adverse effect (AE) resolves to ≤Grade 1. If a DLT was not resolved within 2 weeks, the patient was discontinued from the study. Patients were non-evaluable and can be replaced if (1) termination was for reasons other than DLT, or (2) the patient received less than 75% (16 of 21 days) of the planned onvansertib doses in Cycle 1 for any reason other than DLT related interruptions. Patients who experienced a DLT that subsequently resolved can be permitted to continue with appropriate onvansertib dose reduction, if the Investigator determined that clinical benefit can be derived.

### Adverse Events, Dose Delays and Dose Modifications)

All adverse events (AE), including expected AE, unexpected AE, and SAE were monitored and reported in accordance with the clinical trial protocol in a timely manner (Table 8). The severity of AEs were graded using the latest version of NCI-CTCAE Version 5.0. For each SAE, the highest severity grade was reported. Onvansertib related AEs were generally reversible, and recoveries were generally experienced within 3 weeks. Examples of onvansertib related AEs can include myelosuppression (e.g., anemia, leukopenia, thrombocytopenia, and neutropenia, including febrile neutropenia), and fatigue. Examples of paclitaxel related AEs can include anaphylaxis and severe hypersensitivity reactions characterized by dyspnea and hypotension requiring treatment, angioedema, and generalized urticaria; bone marrow suppression (primarily neutropenia) and neutrophil nadirs; and severe conduction abnormalities. In Table 8, # means that if an event listed in protocol as expected and not requiring expedited reporting, the event did not need to be reported; * means that for participants enrolled and actively participating in the study or for AEs occurring within 30 days of the last intervention, events must be reported within 1 business day of learning of the event; and & means that an event presenting at a higher severity than what was listed within the protocol and/or current consent as expected would be considered unexpected and reportable.

TABLE 8
DF/HCC REPORTABLE ADVERSE EVENTS (AES)

| | DF/HCC REPORTABLE ADVERSE EVENTS(AES) | | | | |
|---|---|---|---|---|---|
| | Gr. 2 and 3 | Gr. 2 and 3 AE | Gr. 4 AE | Gr. 4 AE | Gr. 5 AE Expected |
| Attribution | AE Expected | Unexpected | Expected^{&} | Unexpected^{&} | or Unexpected |
| Unrelated, | Not required | Not required | 10 calendar | 10 calendar | 24 hours* |
| Unlikely | | | days^{#} | days | |
| Possible, | Not required | 10 calendar | 10 calendar | 10 calendar | 24 hours* |
| Probable, | | days | days^{#} | days | |
| Definite | | | | | |

Appropriate monitoring and supportive care were provided to the patients in accordance with the clinical trial protocol. Toxicity related dose delay can be held for a maximum of 28 days and in accordance with the clinical trial protocol. Toxicity related dose modifications were made in accordance with Table 9 to a maximum of 18 mg/m² of onvansertib and 48 mg/m² of paclitaxel. In Table 9, onvansertib was given orally once daily for 21 consecutive days of every 28-day cycle, while paclitaxel was given intravaneously (IV) once on days 1, 8, and 15 of every 28-day cycle. Intra-patient dose escalation can be permitted in accordance with the clinical trial protocol. Toxicity potentially related to the clinical trial were managed in accordance with the clinical trial protocol. For example, hematologic toxicity, non-hematological toxicity, hepatic toxicity, and neuropathy were managed in accordance with Table 10. A patient can also be taken off the treatment and/or study in accordance with the clinical trial protocol. For hematologic toxicity management in Table 7, superscript “1” means that study treatment should be discontinued if blood counts did not recover to Grade 2 or better within 4 weeks of dose interruption; and superscript “2” means that a new cycle may be initiated for patients with absolute neutrophil count (ANC) resolved to baseline grade or <Grade 2 (≥1000/mm³). For the management of non-hematological toxicity in Table 7, participants experiencing intolerable Grade 2 events can have their study medication held and/or dose reduced at the treating investigator's discretion. For the Grade 3 nausea and vomiting within the exceptions, no routine prophylactic anti-emetic treatment was required at the start of study treatment. However, patients should receive appropriate anti-emetic treatment at the first onset of nausea or vomiting and as required thereafter, in accordance with local treatment practice guidelines.

TABLE 9
DOSE REDUCTIONS

| Original | | |
|---|---|---|
| Dose | | |
| Level | Onvansertib Dose Reduction | Paclitaxel Dose Reduction |
| −1 | 1^{st} Reduction: 3 mg/m² | 1^{st} Reduction: 64 mg/m² |
| | 2^{nd} Reduction: Discontinue | 2^{nd} Reduction: 48 mg/m² |
| | Onvansertib Therapy | |
| 0 | 1^{st} Reduction: 6 mg/m² | 1^{st} Reduction: 64 mg/m² |
| | 2^{nd} Reduction: 3 mg/m² | 2^{nd} Reduction: 48 mg/m² |
| | 3^{rd} Reduction: Discontinue | 3^{rd} Reduction: Discontinue |
| | Onvansertib Therapy | Paclitaxel Therapy |
| 1 | 1^{st} Reduction: 9 mg/m² | 1^{st} Reduction: 64 mg/m² |
| | 2^{nd} Reduction: 6 mg/m² | 2^{nd} Reduction: 48 mg/m² |
| | 3^{rd} Reduction 3 mg/m² | 3^{rd} Reduction: Discontinue |
| | 4^{th} Reduction: Discontinue | Paclitaxel Therapy |
| | Onvansertib Therapy | |

TABLE 10
TOXICITY MANAGEMENT

| | | |
|---|---|---|
| Event Term | CTCAE Grade | Management Guidelines |

HEMATOLOGIC TOXICITY MANAGEMENT

| | | |
|---|---|---|
| Anemia and | ≤2 | No dose adjustment is required |
| Thrombocytopenia | ≥3 | Withhold treatment and initiation of next cycle |
| | | until recovery to Grade ≤2. Resume at next |
| | | lower dose¹. |
| Neutrophil Count Decreased | ≤2 | No dose adjustment is required². |
| (not associated with | | |
| fever/infection) | | |
| Neutrophil Count Decreased | ≥3 | 1^{st} Occurrence: Hold study medications dosing |
| (not associated with | | until ≤Grade 2 or baseline². Resume at the |
| fever/infection) | | same dose level. |
| | | Repeat Occurrence: Hold study medications |
| | | dosing until ≤Grade 2 or baseline. Resume |
| | | with one dose level reduction¹. |
| Febrile Neutropenia | ≥3 | Hold study medications dosing until resolution. |
| | | Resume with one dose level reduction¹. |

MANAGEMENT OF NON-HEMATOLOGICAL TOXICITY

| | |
|---|---|
| ≤2 | No dose adjustment is required. |
| ≥3 | Hold study medication dosing. Upon resolution |
| | to ≤Grade 1, resume dosing with one dose |
| | level reduction. |
| | Exceptions: |
| | Grade 3 nausea, vomiting, diarrhea, or |
| | constipation that was not optimally managed. |
| | In this instance, hold study medication dosing |
| | and initiate appropriate medical management |
| | (e.g. anti-emetics, anti-diarrheals, laxatives). If |
| | symptoms resolve to ≤Grade 1 or baseline |
| | within 48 hours of instituting appropriate care, |
| | participants may resume dosing at the same |
| | dose level or may be dose reduced by one dose |
| | level at the treating investigator's discretion. |
| | ≥Grade 3 laboratory abnormalities that are |
| | judged by the treating investigator to be not |
| | clinically significant. Participants may continue |
| | dosing at the same dose level as judged |
| | appropriate by the treating investigator. |
| | Asymptomatic laboratory abnormalities that |
| | resolve to ≤Grade 1 or baseline within 48 |
| | hours of repletion. Study medication should be |
| | held until abnormalities resolve to ≤Grade 1 or |
| | baseline. Repletion and resumption of study |
| | treatment may occur on the same day. |

LIVER TOXICITY MANAGEMENT

| | | |
|---|---|---|
| AST/ALT elevation | Grade 3 (>5.0- | Hold paclitaxel until recovered to ≤Grade 1. |
| | 20.0 × ULN) | Resume paclitaxel dosing with one dose level |
| | | reduction. |
| | Grade 4 (>20.0 × | Discontinue paclitaxel. |
| | ULN) | |

NEUROPATHY MANAGEMENT

| | | |
|---|---|---|
| Paresthesia/Dysesthesia | Grade 1-2 | No dose adjustment is required. |
| | tolerable | |
| | Grade 2 | Hold Paclitaxel until ≤tolerable Grade 2. |
| | intolerable | Resume paclitaxel dosing with one dose level |
| | | reduction. |
| | Grade 3-4 | Hold Paclitaxel until ≤tolerable Grade 2. |
| | | Resume paclitaxel dosing with one dose level |
| | | reduction. |

### Pharmacological Determination

The Phase 1b and Phase 2 clinical trials were conducted in accordance with the Study Calendar (Table 11). Evaluations were conducted in accordance with the clinical trial protocol. Pharmacokinetic evaluations were collected for Phase 1b patients at the following time points in accordance with the clinical trial protocol: C1D1 (pre-paclitaxel dosing and 1, 2, 3, 4, 8 hours post-paclitaxel dosing), C1D8 (2 hours post-paclitaxel dosing), C1D15 (2 hours post-paclitaxel dosing), and C2D1 (Pre-paclitaxel dosing).

Abbreviations used in Table 11 include AE (adverse event), ALT (alanine aminotransferase), AST (aspartate aminotransferase), CBC (complete blood count), CT (computed tomography), ctDNA (circulating tumor DNA), ECOG (Eastern Cooperative Oncology Group (performance score)), ECG (electrocardiograms), EOT (End-of-Treatment), MRI (magnetic resonance imaging), PK (pharmacokinetic), TNM (tumor, lymph nodes, metastasis), and PBMC (peripheral blood mononuclear cells). In Table 11, medical history included recording of relevant medical history, and TNM stage at diagnosis; prior treatment start and end dates should be recorded (if intolerance, the reason should be recorded); physical examination should include height (at screening only), weight, vital signs, and general physical examination; blood chemistry panel (including blood chemistry and CBC with differential) included sodium, potassium, chloride, bicarbonate, calcium, blood urea nitrogen, creatinine, glucose, albumin, alkaline phosphatase, total bilirubin, AST, ALT (CBC and blood chemistry testing can occur up to 48 hours prior to Days 1, 8 and 15 and all visits had a ±3-day window); blood samples for PK analysis were obtained during Cycle 1 on Days 1, 8, and 15, and Cycle 2 Day 1 of phase 1b part only (samples on C1D1 should be collected pre-dose and 1, 2, 3, 4, and 8 hours post dose; samples on C1D8 and C1D15 should be collected 2 hours post-dose; samples on C2D1 should be collected pre-dose; this applied to Phase 1b only); a baseline biopsy was required after consent in the phase 1b and 2 of the study, when easily accessible (if available, site should confirm access to archival tissue; Biopsy at EOT is optional); blood sample (collected in two 10-mL Streck tubes) for ctDNA assessment should be collected on C1D1, C3D1, and at EOT; for disease assessment, subjects must have imaging of the chest/abdomen/pelvis, preferably with a CT scan, within 4 weeks prior to start, and MRI of the brain (or contrast CT scan of the brain if subjects are unable to undergo MRI) must be obtained in subjects with symptoms suggesting possible central nervous system (CNS) metastatic disease; restaging with CT or MRI should be done every 2 cycles (±1 week) (e.g., C2D15) until EOT and EOT assessments should be conducted within 28 days (±5 days) after the last dose of onvansertib; subjects were given onvansertib orally for 21 consecutive days followed by 7 days off (onvansertib was dosed in the clinic on Days 1, 8, and 15 of cycle 1 and Day 1 of cycle 2, but otherwise taken at home); and blood for PBMCs was collected on C1D1, C1D15, and EOT in four 8 mL CPT tubes.

TABLE 11
STUDY CALENDAR OF THE PHASE 1B AND PHASE 2 TRIAL

| | | | | | | Cycles 2 and | | | | |
|---|---|---|---|---|---|---|---|---|---|---|
| | | Cycle 1 (28-day) | | | | Beyond (28-day) | | | | |
| | Screening | Treatment | | | | Treatment | | | | |
| | (≤28 days | Course | | | | Course | | | End-of- | |
| Assessment/ | prior to | (Day) | | | | (Day) | | | Treatment | |
| Procedure | enrollment) | 1 | 8 | 15 | 21 | 1 | 8 | 15 | (EOT) | EDC Timepoints |
| Informed | X | | | | | | | | | N/A |
| consent | | | | | | | | | | |
| Confirmation | X | | | | | | | | | N/A |
| of all | | | | | | | | | | |
| eligibility | | | | | | | | | | |
| criteria | | | | | | | | | | |
| Medical | X | | | | | | | | X | Baseline |
| history | | | | | | | | | | |
| Prior therapy | X | | | | | | | | | Baseline |
| Physical | X | X | X | X | | X | | X | X | Baseline, Day 1 all |
| examination | | | | | | | | | | cycles, EOT |
| ECOG | X | X | X | X | | X | | X | X | Baseline, Day 1 of |
| performance | | | | | | | | | | every Cycle, EOT |
| status | | | | | | | | | | |
| Concomitant | X | X | X | X | | X | | X | X | N/A |
| medication | | | | | | | | | | |
| review | | | | | | | | | | |
| Adverse | X | X | X | X | | X | | X | X | During all visits |
| Event review | | | | | | | | | | after Baseline |
| Triplicate 12- | X | | | | | X | | | | Baseline, and every |
| lead ECG | | | | | | | | | | 2 Cycles during |
| | | | | | | | | | | treatment |
| Clinical | X | X | X | X | | X | X | X | | Baseline, Days |
| chemistry | | | | | | | | | | 1, 8, 15 of all cycles. |
| CBC with | X | X | X | X | | X | X | X | | Baseline, Days |
| differential | | | | | | | | | | 1, 8, 15 of all cycles |
| Blood | | X | X | X | | X | | | | N/A |
| samples for | | | | | | | | | | |
| PK | | | | | | | | | | |
| assessment | | | | | | | | | | |
| Biopsy/ | X | | | | | | | | X | Baseline and EOT |
| Archival tumor | | | | | | | | | | |
| tissue | | | | | | | | | | |
| Blood | | X | | | | X | | | X | N/A |
| samples for | | | | | | | | | | |
| ctDNA | | | | | | | | | | |
| Blood | | X | | X | | | | | X | N/A |
| samples for | | | | | | | | | | |
| PBMC^{k} | | | | | | | | | | |
| Disease | X | | | | | | | X | X | Baseline, every 2 |
| assessment | | | | | | | | | | cycles, EOT |
| (radiographic | | | | | | | | | | |
| imaging) | | | | | | | | | | |
| Onvansertib | | X | X | X | | X | X | X | | Day 1 of all cycles |
| | | | | | | | | | | starting with Cycle |
| | | | | | | | | | | 2, EOT |
| Paclitaxel | | X | X | X | | X | X | X | | Day 1, 8, 15 of all |
| | | | | | | | | | | cycles. |

Disease response and progression were evaluated using the new international criteria proposed by the Response Evaluation Criteria in Solid Tumors (RECIST) guideline (version 1.1). Changes in the largest diameter (unidimensional measurement) of the tumor lesions and the shortest diameter in the case of malignant lymph nodes were used in the RECIST criteria.

Patients with measurable disease present at baseline, who had received at least one cycle of therapy, and had their disease re-evaluated, were evaluated for target disease response. Patients with evaluable lesions at baseline but not meeting the definitions of measurable disease, who received at least one cycle of therapy, and who had their disease re-evaluated, were evaluated for non-target disease.

Response assessments were based on the presence, absence, or unequivocal progression of the lesions. Exemplary responses for patients with a measurable disease can be found in Table 12. In Table 12, the measurable disease can be a target disease. In Table 12, * means that the response confirmation was only for non-randomized trials with response as primary endpoint; and ** means that in exceptional circumstances, unequivocal progression in non-target lesions may be accepted as disease progression. Moreover, in Table 12, participants with a global deterioration of health status requiring discontinuation of treatment without objective evidence of disease progression at that time should be reported as “symptomatic deterioration.” Every effort should be made to document the objective progression even after discontinuation of treatment. Exemplary responses for patients with a non-measurable disease can be found in Table 13. In Table 13, the non-measurable disease can be a non-target disease. In Table 13, “non-CR/non-PD” was preferred over “stable disease” for non-target disease, since SD was increasingly used as an endpoint for assessment of efficacy in some trials. Thus, assigning to this category when no lesions can be measured was not advised.

TABLE 12
THERAPEUTIC FINDING FOR PARTICIPANTS WITH MEASURABLE DISEASE

| Target | | New | Overall | Best Overall Response when |
|---|---|---|---|---|
| Lesions | Non-Target Lesions | Lesions | Response | Confirmation is Required |
| CR | CR | No | CR | ≥4 wks Confirmation* |
| CR | Non-CR/Non-PD | No | PR | ≥4 wks Confirmation* |
| CR | Not evaluated | No | PR | |
| PR | Non-CR/Non-PD/not evaluated | No | PR | |
| SD | Non-CR/Non-PD/not evaluated | No | SD | Documented at least once ≥4 |
| | | | | wks from baseline* |
| PD | Any | Yes or No | PD | no prior SD, PR or CR |
| Any | PD** | Yes or No | PD | |
| Any | Any | Yes | PD | |

TABLE 13
THERAPEUTIC FINDING FOR PARTICIPANTS
WITH NON-MEASURABLE DISEASE

| Non-Target Lesions | New Lesions | Overall Response |
|---|---|---|
| CR | No | CR |
| Non-CR/non-PD | No | Non-CR/non-PD* |
| Not all evaluated | No | not evaluated |
| Unequivocal PD | Yes or No | PD |
| Any | Yes | PD |

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of treating cancer, the method comprising: administrating a microtubule depolymerization (MTDP) inhibitor and a Polo-like kinase 1 (PLK1) inhibitor to a subject with cancer, thereby inhibiting or reducing progression of the cancer in the subject.

2. The method of claim 1, wherein the subject has hematologic or solid cancer.

3. The method of claim 1, wherein the cancer is breast cancer, wherein the breast cancer is optionally invasive breast cancer, unresectable breast carcinoma, locally advanced breast cancer, metastatic breast cancer, inflammatory breast cancer, triple negative breast cancer (TNBC), hormone receptor/growth factor receptor-negative breast cancer, HER2-negative breast cancer, hormone receptor negative breast carcinoma, or a combination thereof.

4. The method of any one of claims 1-3, wherein the subject has TNBC.

5. The method of any one of claims 1-4, comprising identifying the subject with cancer as a subject having more than about 1000 neutrophils per mm³, more than about 100,000 per mm³ platelets, about 1.5 times institutional upper limit normal of total bilirubin, and either less than about 1.5 times institutional upper limit normal or 60 mL/min glomerular filtration rate of serum creatinine, or a combination thereof.

6. The method of any one of claims 1-5, wherein the subject has more than about 1000 neutrophils per mm³, or more than about 100,000 per mm³ platelets.

7. The method of any one of claims 1-5, wherein the subject has about 1.5 times institutional upper limit normal of total bilirubin, and/or has less than about 1.5 times institutional upper limit normal or 60 mL/min glomerular filtration rate of serum creatinine.

8. The method of any one of claims 1-7, wherein the PLK1 inhibitor and the MTDP inhibitor are co-administered simultaneously.

9. The method of any one of claims 1-7, wherein the PLK1 inhibitor and the MTDP inhibitor are administered sequentially.

10. The method of any one of claims 1-9, wherein the administration of the PLK1 inhibitor is orally, and optionally the subject fasts for more than about 30 minutes prior to the administration and/or the subject fasts for about 4 hours after the administration.

11. The method of any one of claims 1-10, wherein the administration of the MTDP inhibitor is intravenously, optionally through an in-line filter with a microporous membrane not greater than 0.22 micron, and further optionally wherein prior to administration, the subject is administered (i) about 20 mg of dexamethasone orally at about 12 hours and about 6 hours prior or about 12 mg of dexamethasone intravenously at about 60 minutes prior; (ii) about 50 mg of diphenhydramine intravenously at from about 30 minutes to 60 about minutes prior; and/or (iii) about 300 mg of cimetidine intravenously at from about 30 minutes to about 60 minutes prior or about 20 mg of famotidine intravenously from about 30 minutes to about 60 minutes prior.

12. The method of any one of claims 1-11, wherein the inhibition of cancer progression is greater than the combined inhibition of progression caused by the MTDP inhibitor alone plus the PLK1 inhibitor alone.

13. The method of any one of claims 1-12, wherein the subject achieves a complete response.

14. The method of any one of claims 1-13, wherein the subject has received a prior MTDP inhibitor or PLK1 inhibitor treatment.

15. The method of any one of claims 1-14, wherein the subject did not respond to treatment with the MTDP inhibitor or PLK1 inhibitor alone.

16. The method of any one of claims 1-14, wherein the subject is known to be resistant to an MTDP inhibitor or PLK1 inhibitor therapy.

17. The method of any one of claims 1-16, wherein the MTDP inhibitor and the PLK1 inhibitor are each administered to the subject in a cycle of 28 days.

18. The method of any one of claims 1-17, wherein the MTDP inhibitor is administered to the subject in a cycle of 28 days and PLK1 inhibitor is administered to the subject in a cycle of 28 days, comprising from about 14-28 days of administration and from about 0-14 days of non-administration, and optionally from about 21 days of administration of the PLK1 inhibitor and from about 7 days of non-administration of the PLK1 inhibitor.

19. The method of any one of claims 1-18, wherein the MTDP inhibitor, the PLK1 inhibitor, or both are administered in a cycle of 28 days.

20. The method of any one of claims 1-16, wherein each cycle of treatment is at least about 28 days.

21. The method of any one of claims 1-16, wherein each cycle of treatment is from about 14 days to about 28 days.

22. The method of any one of claims 1-21, wherein the PLK1 inhibitor is administered on at least seven days, at least fourteen days, or at least twenty-one days in a cycle.

23. The method of any one of claims 1-21, wherein the PLK1 inhibitor is not administered on at least one day, at least three days, or at least seven days in a cycle.

24. The method of any one of claims 1-23, wherein the MTDP inhibitor is administered once or twice weekly.

25. The method of any one of claims 1-23, wherein the MTDP inhibitor is administered once weekly for two or three consecutive weeks in a cycle.

26. The method of any one of claims 1-25, wherein the subject undergoes at least two cycles of the administration of the MTDP inhibitor and the PLK1 inhibitor.

27. The method of any one of claims 1-26, wherein the MTDP inhibitor is paclitaxel, docetaxel, acetyltaxol, paclitaxel; lutetium Lu 177 vipivotide tetraxetan; 7-hexanoyltaxol; cabazitaxel; larotaxel; milataxel; ortataxel; tesetaxel; taxoprexin; opaxio; taxoprexin (DHA-paclitaxel); Poly(L-glutamic acid)-paclitaxel; abraxane; SB-T-1214; SB-T1216; SB-T121602; SB-T-12854; DHA-SB-T1214; abeo-taxanes, wherein the abeo-taxanes is optionally abeo-taxane 15a.2; docetaxl-d9-t-Boc; docetaxel-f3-t-Boc; cabazitaxel-7,10-d₆; Poly(glutamyl-glutamate)-taxane conjugates; or a pharmaceutically acceptable salt, hydrate, solvate or prodrug thereof; or any combinations thereof.

28. The method of any one of claims 1-26, wherein MTDP inhibitor is paclitaxel.

29. The method of any one of claims 1-28, wherein from about 48 mg/m² of body surface area to about 80 mg/m² of body surface area of paclitaxel is administered to the subject.

30. The method of any one of claims 1-29, wherein the PLK1 inhibitor is onvansertib (NMS-P937), BI2536, volasertib (BI 6727), GSK461364, adavosertib (AZD1775), CYC140, HMN-176, HMN-214, rigosertib (ON-01910), MHLN0905, TKM-080301, TAK-960, GTPL10072, Ro3280; or a pharmaceutically acceptable salt, hydrate, solvate or prodrug thereof; and any combinations thereof, optionally wherein the PLK1 inhibitor is onvansertib.

31. The method of claim 30, wherein onvansertib is administered at a dose from about 6 mg/m² of body surface area to about 24 mg/m² of body surface area, optionally at a dose of 9 mg/m² of body surface area, 12 mg/m² of body surface area or 18 mg/m² of body surface area.

32. The method of any one of claims 1-31, wherein the subject has received at least one prior cancer treatment.

33. The method of claim 32, where the prior treatment does not comprise the use of a MTDP inhibitor, a PLK1 inhibitor, or both; and optionally the PLK1 inhibitor is onvansertib.

34. The method of any one of claims 1-33, wherein the subject was in remission for cancer.

35. The method of any one of claims 1-34, wherein the subject in remission for cancer was in complete remission (CR) or in partial remission (PR).

36. The method of any one of claims 1-35, further comprising determining cancer status of the subject.

37. The method of any one of claims 1-36, further comprising determining responsiveness of the subject to the treatment of the MTDP inhibitor and the PLK1 inhibitor.

38. The method of any one of claims 1-37, further comprising administering one or more cancer therapeutics or therapies for the cancer.

39. The method of any one of claims 1-38, the subject is human.

40. A method of sensitizing cancer cells to a microtubule depolymerization (MTDP) inhibitor, the method comprising: contacting cancer cells with a composition comprising a Polo-like kinase 1 (PLK1) inhibitor, thereby sensitizing the cancer cells to the MTDP inhibitor; optionally wherein the PLK1 inhibitor is onvansertib and/or MTDP inhibitor is paclitaxel.

41. The method of claim 40, wherein contacting cancer cells with the composition occurs in vitro, ex vivo, and/or in vivo.

42. The method of any one of claims 40-41, wherein contacting cancer cells with the composition is in a subject; optionally wherein the subject did not respond to, or is known to be resistant to, the MTDP inhibitor or another MTDP inhibitor.

43. The method of any one of claims 40-42, wherein the subject had prior treatment with the MTDP inhibitor or another MTDP inhibitor.

44. The method of any one of claims 40-43, wherein the subject is a mammal, and optionally the mammal is human.

45. The method of any one of claims 40-44, comprising determining sensitization of the cancer cells to the MTDP inhibitor after being contacted with the composition.

46. The method of any one of claims 40-45, comprising contacting the cancer cells with the MTDP inhibitor; optionally wherein contacting the cancer cells with the MTDP inhibitor occurs in the subject.

47. The method of any one of claims 40-46, comprising determining the response of the subject to the MTDP inhibitor.

48. The method of any one of claims 40-47, wherein contacting the cancer cells with the MTDP inhibitor is concurrent with the contacting the cancer cells with the composition, or after the contacting the cancer cells with the composition.

49. The method of any one of claims 40-48, wherein the subject having more than about 1000 neutrophils per mm³, or more than about 100,000 per mm³ platelets.

50. A kit, comprising:
a polo-like kinase 1 (PLK1) inhibitor; and
a manual providing instructions for co-administrating the PLK1 inhibitor with a microtubule depolymerization (MTDP) inhibitor to a subject in need thereof for treating cancer.

51. The kit of claim 50, wherein the cancer is hematologic or solid cancers, and optionally the cancer is breast cancer, wherein the breast cancer is optionally invasive breast cancer, unresectable breast carcinoma, locally advanced breast cancer, metastatic breast cancer, inflammatory breast cancer, triple negative breast cancer (TNBC), hormone receptor/growth factor receptor-negative breast cancer, HER2-negative breast cancer, hormone receptor negative breast carcinoma, or a combination thereof; optionally wherein the cancer is TNBC.

52. The kit of any one of claims 50-51, wherein the subject has more than about 1000 neutrophils per mm³, more than about 100,000 per mm³ platelets, about 1.5 times institutional upper limit normal of total bilirubin, and either less than about 1.5 times institutional upper limit normal or 60 mL/min glomerular filtration rate of serum creatinine.

53. The kit of any one of claims 50-52, wherein the PLK1 inhibitor is onvansertib and/or the MTDP inhibitor is paclitaxel.

54. The kit of any one of claims 50-53, wherein the instructions comprise instructions for co-administrating the PLK1 inhibitor and the MTDP inhibitor simultaneously.

55. The kit of any one of claims 50-54, wherein the instructions comprise instructions for co-administrating the PLK1 inhibitor and the MTDP inhibitor sequentially.

56. The kit of any one of claims 50-55, wherein the instructions comprise instructions for administering of the PLK1 inhibitor orally, wherein prior to the administration, the subject fasts for more than about 30 minutes, and wherein after the administration, the subject fasts for about 4 hours.

57. The kit of any one of claims 50-56, wherein the instructions comprise instructions for administrating the MTDP inhibitor intravenously, optionally through an in-line filter with a microporous membrane not greater than 0.22 micron, and optionally prior to administration, the subject is administered (i) about 20 mg of dexamethasone orally at about 12 hours and about 6 hours prior or about 12 mg of dexamethasone intravenously at about 60 minutes prior; (ii) about 50 mg of diphenhydramine intravenously at from about 30 minutes to 60 about minutes prior; and/or (iii) about 300 mg of cimetidine intravenously at from about 30 minutes to about 60 minutes prior or about 20 mg of famotidine intravenously from about 30 minutes to about 60 minutes prior.

58. The kit of any one of claims 50-57, wherein the instructions comprise instructions for administering to a subject that has received a prior MTDP inhibitor or PLK1 inhibitor treatment.

59. The kit of any one of claims 50-58, wherein the instructions comprise instructions for administering to a subject that did not respond to treatment with the MTDP inhibitor or PLK1 inhibitor alone.

60. The kit of any one of claims 50-59, wherein the instructions comprise instructions for administering to a subject known to be resistant to an MTDP inhibitor or PLK1 inhibitor therapy.

61. The kit of any one of claims 50-60, wherein the instructions comprise instructions for administering each of the paclitaxel and onvansertib to the subject in a cycle of 28 days.

62. The kit of any one of claims 50-61, wherein the instructions comprise instructions for administering each of the paclitaxel and onvansertib to the subject in a cycle of at least five times within a week.

63. The kit of any one of claims 50-62, wherein the instructions comprise instructions for administering the MTDP inhibitor, onvansertib, or both are in a cycle of at least 7 days.

64. The kit of claim 63, wherein each cycle of treatment is at least about 21 days, or is from about 14 days to about 28 days.

65. The kit of any one of claims 50-64, wherein the instructions comprise instructions for administering onvansertib daily for 14-21 consecutive days in a cycle of 28 days.

66. The kit of any one of claims 50-65, wherein the instructions comprise instructions for not administering onvansertib on three, five or seven consecutive days in the cycle.

67. The kit of any one of claims 50-66, wherein the instructions comprise instructions for administrating the MTDP inhibitor weekly for three consecutive weeks in a cycle.

68. The kit of any one of claims 50-67, wherein the MTDP inhibitor is a reversible MTDP inhibitor.

69. The kit of any one of claims 50-68, wherein the MTDP inhibitor is paclitaxel or a pharmaceutically acceptable salt, hydrate, solvate or prodrug thereof.

70. The kit of any one of claims 50-69, wherein the instructions comprise instructions for administering onvansertib at a dose of from about 3 mg/m² of body surface area to about 24 mg/m² of body surface area and/or administering paclitaxel at a dose of from about 48 mg/m² of body surface area to about 80 mg/m² of body surface area, optionally wherein the instructions comprise instructions for administering onvansertib at a dose of 9 mg/m² of body surface area, 12 mg/m² of body surface area or 18 mg/m² of body surface area.

71. The kit of any one of claims 50-70, wherein the subject has received at least one prior treatment for the cancer.

72. The kit of claim 71, where the prior treatment does not comprise the use of a MTDP inhibitor, onvansertib, or both.

73. The kit of any one of claims 50-72, wherein the subject was in remission for cancer.

74. The kit of claim 73, wherein the subject in remission for cancer was in complete remission (CR) or in partial remission (PR).

75. The kit of any one of claims 50-74, further comprising the MTDP inhibitor, preferably paclitaxel; and/or PLK1 inhibitor, preferably onvansertib.
